SEQUENCE LISTING (3 sequences)

SEQ ID NO: 1 - DNA, 48 bp, synthetic construct
  source 1..48: mol_type = other DNA
  modified_base 28: mod_base = OTHER; note = 7-(2-thienyl)imidazo[4,5-b]pyridine
  modified_base 39: mod_base = OTHER; note = 7-(2-thienyl)imidazo[4,5-b]pyridine
cccgcccgggtccgcgaagcggtaggtntgggctaggcngctggcggg

SEQ ID NO: 2 - DNA, 55 bp, synthetic construct
  source 1..55: mol_type = other DNA
  modified_base 28: mod_base = OTHER; note = 7-(2-thienyl)imidazo[4,5-b]pyridine
  modified_base 39: mod_base = OTHER; note = 7-(2-thienyl)imidazo[4,5-b]pyridine
cccgcccgggtccgcgaagcggtaggtntgggctaggcngctggcgggcgcgaag

SEQ ID NO: 3 - DNA, 57 bp, synthetic construct
  source 1..57: mol_type = other DNA
  modified_base 28: mod_base = OTHER; note = 7-(2-thienyl)imidazo[4,5-b]pyridine
  modified_base 39: mod_base = OTHER; note = 7-(2-thienyl)imidazo[4,5-b]pyridine
  misc_difference 53: note = any base
cccgcccgggtccgcgaagcggtaggtntgggctaggcngctggcgggcgcgnagcg

(19) United States
(12) Patent Application Publication
(10) Pub. No.: US 2026/0265761 A1
(43) Pub. Date: Sep. 10, 2026

(54) TOPICAL COMPOSITIONS CONTAINING APTAMER AND IONIC LIQUID

(71) Applicant: CAGE BIO INC., San Carlos, CA (US)

(72) Inventors: Nitin JOSHI, San Carlos, CA (US); Chase Corley, San Carlos, CA (US); Abhirup Mandel, San Carlos, CA (US)

(21) Appl. No.: 19/669,980

(22) Filed: May 6, 2026

Related U.S. Application Data
(63) Continuation of application No. PCT/US25/51742, filed on Oct. 20, 2025.
(60) Provisional application No. 63/709,377, filed on Oct. 18, 2024.
(60) Provisional application No. 63/727,624, filed on Dec. 3, 2024.

(51) Int. Cl. C12N 15/115; A61K 9/00; A61K 47/02; A61K 47/10; A61K 47/12; A61K 47/18; A61K 47/38; A61P 17/14

(52) U.S. Cl. CPC C12N 15/115; A61K 9/0014; A61K 47/02; A61K 47/10; A61K 47/12; A61K 47/186; A61K 47/38; A61P 17/14; C12N 2310/16

(57) ABSTRACT

One aspect of the present disclosure relates to a topical composition, comprising aptamer or a pharmaceutically acceptable salt thereof, and an ionic liquid in an amount sufficient to allow a therapeutically effective amount of aptamer to reach a target depth within or beyond the skin, wherein the ionic liquid comprises a choline cation and a fatty acid anion. Another aspect of the present disclosure relates to a method of delivering a therapeutic DNA oligonucleotide, such as an aptamer, to or through a skin.

### CROSS-REFERENCE TO RELATED APPLICATIONS

The present application is a US National Stage of International Patent Application PCT/US2025/051742, filed on Oct. 20, 2025, which claims priority to U.S. Provisional Patent Application 63/709,377, filed on Oct. 18, 2024 and U.S. Provisional Patent Application 63/727,624 filed on Dec. 3, 2024. The prior applications are considered part of the disclosure of the present application and are incorporated herein by reference in their entirety.

### SEQUENCE LISTING

The instant application contains a Sequence Listing which has been submitted electronically in .XML format and is hereby incorporated by reference in its entirety. Said copy has the file name “128298-0014WO01.xml”, is 7,514 bytes in size, and has a production date of Jan. 3, 2026.

### BACKGROUND OF THE DISCLOSURE

Alopecia areata is a widespread hair loss condition that occurs widely regardless of age or gender, and frequently occurs on the head. Recent studies have revealed that autoimmune abnormalities are involved in the pathogenesis of alopecia areata. Normal hair follicle tissues and the surrounding areas are in an immune-tolerant environment, with little or no expression of MHC class I and II. In contrast, in alopecia areata lesions, expression of MHC class I and II increases and immune tolerance of hair follicles is broken.

Vitiligo is an acquired pigmentary skin disorder caused by the absence of pigmentary cells from the epidermis that results in white macules and patches on the body. The condition is usually associated with a few autoimmune disorders, with thyroid abnormalities being the most common one. The etiology of vitiligo is unknown but there are different theories to explain its pathogenesis. Vitiligo presents clinically with signs and symptoms of white spots on the body distributed symmetrically and more obvious in people with dark skin. The lesions are characterized by well-demarcated pearly white or depigmented macules and patches, oval, round, or linear-shaped, and the borders are convex, ranging from the size of a few millimeters to centimeters, and enlarged centrifugally. There are different clinical variants of vitiligo, which are trichrome, marginal inflammatory, and quadrichrome vitiligo. Koebner phenomenon (Development of vitiligo at specific trauma prone sites, like cut, burn, or abrasion) is also a common clinical manifestation. Initial lesions occur most frequently on the hands, forearms, feet, and face, favoring a periocular or perioral distribution. On the basis of the distribution pattern Vitiligo is classified into three types: generalized, segmental, and localized. The severity of the disease is scored by the body surface area affected. The course of the disease is often unpredictable and varies in response to the treatment. Depigmentation is often the cause of psychological distress, social stigmatization, and low self-esteem.

### SUMMARY OF THE DISCLOSURE

Disclosed herein, in certain embodiments, is a topical composition, comprising aptamer or a pharmaceutically acceptable salt thereof, and an ionic liquid in an amount sufficient to allow a therapeutically effective amount of aptamer to reach a target depth within or beyond the skin, wherein the ionic liquid comprises a choline cation and a fatty acid anion. In some embodiments, the ionic liquid is choline geranate. In some embodiments, the topical composition is a gel, such as a hydrogel.

Disclosed herein, in certain embodiments, is a method of delivering a therapeutic DNA oligonucleotide, such as an aptamer, to or through a skin. Another aspect of the present disclosure is directed to a topical composition, comprising aptamer or a pharmaceutically acceptable salt thereof, and an ionic liquid in an amount sufficient to allow a therapeutically effective amount of aptamer to reach a target depth within or beyond the skin.

Disclosed herein, in certain embodiments, is a method of locally inhibiting expression of MHC class I and/or MHC class II within an epidermis layer, a dermis layer, a subcutaneous tissue layer, or a muscle tissue. Another aspect of the present disclosure is directed to a method of locally restoring or improving immune tolerance of hair follicles.

Disclosed herein, in certain embodiments, is a method of delivering aptamer to or through a skin, the method comprising administering to the skin a pharmaceutical composition comprising aptamer or a pharmaceutically acceptable salt thereof, and an ionic liquid in an amount sufficient to allow a therapeutically effective amount of aptamer to reach a target depth within or beyond the skin, wherein the ionic liquid comprises a choline cation and a fatty acid anion.

Disclosed herein, in certain embodiments, is a method of locally inhibiting IFN-γ awithin an epidermis layer, a dermis layer, a subcutaneous tissue layer, or a muscle tissue, the method comprising administering to the skin a pharmaceutical composition comprising aptamer or a pharmaceutically acceptable salt thereof, and an ionic liquid in an amount sufficient to allow a therapeutically effective amount of aptamer to reach a target depth within or beyond the skin, wherein the ionic liquid comprises a choline cation and a fatty acid anion.

Disclosed herein, in certain embodiments, is a method of locally inhibiting expression of MHC class I and/or MHC class II within an epidermis layer, a dermis layer, a subcutaneous tissue layer, or a muscle tissue, the method comprising administering to the skin a pharmaceutical composition comprising aptamer or a pharmaceutically acceptable salt thereof, and an ionic liquid in an amount sufficient to allow a therapeutically effective amount of aptamer to reach a target depth within or beyond the skin, wherein the ionic liquid comprises a choline cation and a fatty acid anion.

Disclosed herein, in certain embodiments, is a method of locally restoring or improving immune tolerance of hair follicles within an epidermis layer, a dermis layer, a subcutaneous tissue layer, or a muscle tissue, the method comprising administering to the skin a pharmaceutical composition comprising aptamer or a pharmaceutically acceptable salt thereof, and an ionic liquid in an amount sufficient to allow a therapeutically effective amount of aptamer to reach a target depth within or beyond the skin, wherein the ionic liquid comprises a choline cation and a fatty acid anion.

Disclosed herein, in certain embodiments, is a method of locally restoring or improving immune tolerance of hair follicles within an epidermis layer, a dermis layer, a subcutaneous tissue layer, or a muscle tissue, the method comprising administering to the skin a pharmaceutical composition comprising aptamer or a pharmaceutically acceptable salt thereof, and an ionic liquid in an amount sufficient to allow a therapeutically effective amount of aptamer to reach a target depth within or beyond the skin, wherein the ionic liquid comprises a choline cation and a fatty acid anion.

### DESCRIPTION OF DRAWINGS

FIG. 1 shows a change in the number of hairs on a grafted skin tissue piece before and after administration of an aptamer according to an embodiment of the present disclosure. The vertical axis shows the change in the number of hairs per grafted skin tissue piece.

FIG. 2A shows an inhibition of MHC class I expression in the dermal sheath cup by administration of an aptamer according to an embodiment of the present disclosure.

FIG. 2B shows an inhibition of MHC class I expression in the outer root sheath by administration of the DNA oligonucleotide according to an embodiment of the present disclosure.

FIG. 3A shows an inhibition of MHC class II expression in the connective tissue sheath by administration of the DNA oligonucleotide according to an embodiment of the present disclosure.

FIG. 3B shows an inhibition of MHC class II expression in the outer root sheath by administration of the DNA oligonucleotide according to an embodiment of the present disclosure.

FIG. 4A shows a skin flux assay of ionic liquid compositions containing an exemplary DNA aptamer, showing 2% ionic liquid (choline geranate) providing better dermal bioavailability than 10% ionic liquid (choline geranate).

FIG. 4B shows a skin flux assay of ionic liquid compositions containing an exemplary DNA aptamer, showing good dermal bioavailability at various aptamer concentrations.

FIG. 5 shows design of a clinical study of using topical composition CGB-600 on patients of alopecia areata and patients of vitiligo.

FIG. 6 illustrate an example of an alopecia areata patient in the clinical study, before and after application of CGB-600.

FIG. 7 illustrate an example of a vitiligo patient in the clinical study, before and after application of CGB-600.

FIG. 8 illustrate an example of another vitiligo patient in the clinical study, before and after application of CGB-600.

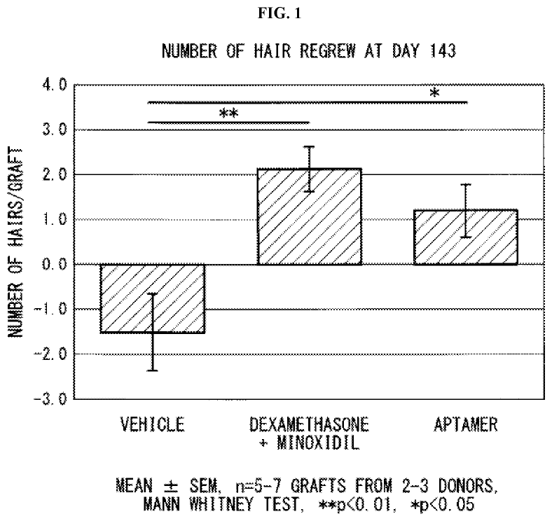

### DETAILED DESCRIPTION OF THE DISCLOSURE

Disclosed herein, in certain embodiments, is a topical composition, comprising aptamer or a pharmaceutically acceptable salt thereof, and an ionic liquid in an amount sufficient to allow a therapeutically effective amount of aptamer to reach a target depth within or beyond the skin, wherein the ionic liquid comprises a choline cation and a fatty acid anion.

### Certain Terminology

The terminology used herein is for the purpose of describing particular cases only and is not intended to be limiting. The below terms are discussed to illustrate meanings of the terms as used in this specification, in addition to the understanding of these terms by those of skill in the art. As used herein and in the appended claims, the singular forms “a”, “an”, and “the” include plural referents unless the context clearly dictates otherwise. It is further noted that the claims can be drafted to exclude any optional element. As such, this statement is intended to serve as antecedent basis for use of such exclusive terminology as “solely,” “only” and the like in connection with the recitation of claim elements, or use of a “negative” limitation.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating un-recited number may be a number which, in the context in which it is presented, provides the substantial equivalent of the specifically recited number. Where a range of values is provided, it is understood that each intervening value, to the tenth of the unit of the lower limit unless the context clearly dictates otherwise, between the upper and lower limit of that range and any other stated or intervening value in that stated range, is encompassed within the methods and compositions described herein are. The upper and lower limits of these smaller ranges may independently be included in the smaller ranges and are also encompassed within the methods and compositions described herein, subject to any specifically excluded limit in the stated range. Where the stated range includes one or both of the limits, ranges excluding either or both of those included limits are also included in the methods and compositions described herein.

The terms “individual,” “patient,” or “subject” are used interchangeably. None of the terms require or are limited to situation characterized by the supervision (e.g. constant or intermittent) of a health care worker (e.g. a doctor, a registered nurse, a nurse practitioner, a physician's assistant, an orderly, or a hospice worker). Further, these terms refer to human or animal subjects.

“Treating” or “treatment” refers to both therapeutic treatment and prophylactic or preventative measures, wherein the object is to prevent or slow down (lessen) a targeted pathologic condition or disorder. Those in need of treatment include those already with the disorder, as well as those prone to have the disorder, or those in whom the disorder is to be prevented. For example, a subject or mammal is successfully “treated” for rosacea, if, after receiving a therapeutic amount of a composition according to the methods of the present disclosure, the subject shows observable and/or measurable reduction in or absence of one or more of the following: reduction in the erythema; reduction in the appearance of red veins; papules, and pustules.

The terms “effective amount” or “therapeutically effective amount,” as used herein, refer to a sufficient amount of an agent or a compound being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition including a compound as disclosed herein required to provide a clinically significant decrease in disease symptoms without undue adverse side effects. An appropriate “effective amount” in any individual case may be determined using techniques, such as a dose escalation study. The term “therapeutically effective amount” includes, for example, a prophylactically effective amount. An “effective amount” of a compound disclosed herein is an amount effective to achieve a desired pharmacologic effect or therapeutic improvement without undue adverse side effects. It is understood that “an effect amount” or “a therapeutically effective amount” can vary from subject to subject, due to variation in metabolism of the compound, age, weight, general condition of the subject, the condition being treated, the severity of the condition being treated, and the judgment of the prescribing physician. By way of example only, therapeutically effective amounts may be determined by routine experimentation, including but not limited to a dose escalation clinical trial.

Unless defined otherwise, all technical and scientific terms used herein have the same meaning as commonly understood by one of ordinary skill in the art to which the methods and compositions described herein belong. Although any methods and materials similar or equivalent to those described herein can also be used in the practice or testing of the methods and compositions described herein, representative illustrative methods and materials are now described.

### DNA Oligonucleotide/Aptamer

It is contemplated herein that inhibitors of IFN-γ activity are promising as therapeutic agents for autoimmune diseases. Previously, antibodies and JAK inhibitors have been developed as agents that inhibit IFN-γ activity. However, the present disclosure recognizes anti-IFN-γ antibodies, for example, have some problems, including that (1) since they are biologics, there is a risk of biological contamination and the like, (2) in case of heterologous antibodies, antigenicity becomes a problem in long-term administration, and (3) since they are protein preparations, a cold chain is required for storage and transportation.

JAK inhibitors have been applied to and marketed for rheumatoid arthritis, an autoimmune disease. The present disclosure recognizes that multiple subtypes of JAK exist and are activated by binding to the intracellular domains of multiple cytokine receptors including interleukin 2 (IL-2) receptor, interleukin 4 (IL-4) receptor, interleukin 7 (IL-7) receptor, and interferon-α (IFN-α) receptor, as well as the IFN-γ receptor, and transmit receptor signals. As a result, JAK inhibitors may inhibit signaling of not only IFN-γ but also IL-2, IL-4, IL-7, IFN-α, and the like (Yvan Jamilloux, et. al., Autoimmunity Reviews, 2019, 18, 102390). This raises safety concerns for long-term administration and suggests a possibility of developing unanticipated adverse effects.

Without wishing to be bound by any particular theory, the present disclosure relates to topical compositions containing an ionic liquid and a DNA oligonucleotide, such as an aptamer. In some embodiment, the topical composition delivers the therapeutic DNA oligonucleotide, such as the aptamer, to the therapeutic site within or beyond the skin. In some embodiment, the locally delivered aptamer selectively inhibits IFN-γ without significant risk of biological contamination, antigenicity in long-term administration, and/or stability or storage issues.

In order to solve the problems described above, the autoimmune disease therapeutic agent containing as an active ingredient an oligonucleotide that selectively binds to IFN-γ and the oligonucleotide of the present disclosure adopt the following aspects.

The first aspect of the present disclosure provides an autoimmune disease therapeutic agent containing as an active ingredient a DNA oligonucleotide having a nucleotide sequence set forth in any of SEQ ID NO 1-3 and selectively binding to interferon-γ (IFN-γ). The DNA oligonucleotide of this aspect exerts a therapeutic effect on autoimmune diseases by selectively binding to IFN-γ.

The nucleotide sequence set forth in SEQ ID NO:2 is a sequence in which an oligonucleotide comprising natural bases of 9 residues is added to a 3′ end of the nucleotide sequence set forth in SEQ ID NO: 1.

The nucleotide sequence set forth in SEQ ID NO:3 is a sequence in which a 53rd base from the 5′ end of the nucleotide sequence set forth in SEQ ID NO:2 was replaced with any base.

In the first aspect described above, a base X in a sequence of the DNA oligonucleotide having the nucleotide sequence set forth in SEQ ID NO:3 may be an artificially produced base, and the artificially produced base may be chemically modified with a low-molecular-weight compound.

The low-molecular-weight compound in the first aspect described above may be an anti-inflammatory compound selected from glucocorticoid, tacrolimus, sirolimus, cyclosporine, methotrexate and leflunomide, or an antibiotic selected from erythromycin, azithromycin, kanamycin, ofloxacin, gatifloxacin, tetracycline and vancomycin.

In the first aspect described above, a base X in a sequence of the DNA oligonucleotide having the nucleotide sequence set forth in SEQ ID NO:3 may be an artificially produced base, and the artificially produced base may be chemically modified with a medium-molecular-weight compound, a high-molecular-weight compound, a biopolymer, or a biocompatible polymer.

The medium-molecular-weight compound in the first aspect described above may have a molecular weight of about 1000 to 20000. The high-molecular-weight compound of the present aspect may have a molecular weight of about 20000 to 400000.

The high-molecular-weight compound in the first aspect described above may be any biocompatible large molecule with a molecular weight of 20000 or more. Examples of medium-molecular-weight compounds or high-molecular-weight compounds in the present aspect include, but are not limited to, polyethylene glycols (PEGs), bipolar polymers, oligosaccharides, fat-soluble polymers, peptides, oligonucleotides, and antibodies. Antibodies belong to high-molecular-weight compounds, while PEGs, bipolar polymers, oligosaccharides, fat-soluble polymers, peptides, and oligonucleotides belong to medium-molecular-weight compounds or high-molecular-weight compounds, depending on their molecular weight. The molecular weight of a medium-molecular-weight compound or a high-molecular-weight compound is expressed as an average molecular weight defined by the number average molecular weight (Mn) or the weight average molecular weight (Mw).

In the first aspect described above, the autoimmune disease to be treated may be an autoimmune skin disease selected from the group consisting of alopecia areata, vitiligo vulgaris, psoriasis, scleroderma, dermatomyositis, atopic dermatitis, cutaneous lupus erythematosus and IgA-related dermatitis.

In the first aspect described above, the autoimmune disease to be treated may be an autoimmune disease in the bladder selected from the group consisting of interstitial cystitis and bladder pain syndrome.

In the first aspect described above, the autoimmune disease to be treated may be an autoimmune disease in the eye selected from the group consisting of uveitis, dry eye, keratitis sicca, episcleritis and scleritis.

In the first aspect described above, the autoimmune disease to be treated may be a systemic autoimmune disease selected from the group consisting of multiple sclerosis, systemic lupus erythematosus, endometriosis, myocarditis, type I diabetes mellitus, thyroiditis, premature ovarian failure, Sjogren syndrome, Raynaud syndrome, rheumatoid arthritis, myasthenia gravis, Takayasu arteritis, Addison disease, Guillain-Barre syndrome, hypothyroidism, sarcoidosis, hemophagocytic lymphohistiocytosis, thrombotic thrombocytopenia purpura, and autoimmune hepatitis.

In the first aspect of the present disclosure described above, the autoimmune disease to be treated may be an autoimmune disease of the digestive organs selected from the group consisting of Crohn disease, ulcerative colitis, autoimmune pancreatitis, biliary cholangitis, and autoimmune atrophic gastritis.

The second aspect of the present disclosure provides a DNA oligonucleotide having a nucleotide sequence set forth in any of SEQ ID NO:1 to 3 and having a therapeutic effect on autoimmune diseases by selectively binding to interferon-γ (IFN-γ). The DNA oligonucleotide of this aspect exerts a therapeutic effect on autoimmune diseases by selectively binding to IFN-γ. The nucleotide sequence set forth in SEQ ID NO:2 is the nucleotide sequence set forth in SEQ ID NO: 1 having an oligonucleotide of 9 natural base residues added to the 3′ end. The nucleotide sequence set forth in SEQ ID NO:3 is a sequence in which the 53rd base counted from the 5′ end of the nucleotide sequence set forth in SEQ ID NO:2 was changed to any base X, and the X is any natural base or artificially produced base. The artificially produced base of this aspect in a sequence of the DNA oligonucleotide having a nucleotide sequence set forth in SEQ ID NO:3 may be chemically modified with the low-molecular-weight compound, the medium-molecular-weight compound, or the high-molecular-weight compound described above.

The autoimmune diseases for which the autoimmune disease therapeutic agent of the second aspect above is given for the treatment may be the autoimmune skin diseases, the autoimmune diseases in the bladder, the autoimmune diseases in the eye, the systemic autoimmune diseases, and the autoimmune diseases of the digestive organs exemplified above.

### Advantageous Effects of Disclosure

According to the DNA oligonucleotide having a therapeutic effect on autoimmune diseases of the present disclosure, a selective inhibition of IFN-γ is achieved by selectively binding to IFN-γ. Also, according to the DNA oligonucleotide having a therapeutic effect on autoimmune diseases of the present disclosure, its production is made without a risk of biological contamination, since there is no need to use serum or other substances in its production. Further, the DNA oligonucleotide having a therapeutic effect on autoimmune diseases of the present disclosure can be stored at room temperature, which is advantageous compared to conventional methods in terms of transportation and storage costs and can improve convenience for patients who use it.

According to the autoimmune disease therapeutic agent containing the DNA oligonucleotide as an active ingredient of the present disclosure, the DNA oligonucleotide of the present disclosure inhibits only the action of IFN-γ, even when administered for a long time, thus reducing unanticipated adverse effects compared to Janus kinase inhibitors and other therapeutic agents. Further, when compared to anti-IFN-γ antibodies, the DNA oligonucleotide of the present disclosure is less antigenic than antibodies, which makes it an agent that can be used for a long period of time. Furthermore, the DNA oligonucleotide of the present disclosure has no risk of biological contamination and can be stored at room temperature.

It is known that overproduction of IFN-γ, which is a factor in the development of alopecia areata, induces expression of major histocompatibility complex (MHC) class II on T cell membranes (Viktor Steimle, et. al, Science, 1994, 265 (5168), 106-109; Carmen Gianfrani, et. al., Journal of Autoimmunity, 2018 (89), 1-10; Giulio Cavalli, et. al., Proc. Natl. Acad. Sci. USA, 2016, 113 (5), 1363-1368), and further inhibits differentiation of regulatory T cells (Treg cells) that control immunity in an inhibitory manner (Susan A. Alalehan, et. al., Sur. J. Immunol., 2015, 45, 988-998). The mechanism above is postulated to be one of the main causes of the development of autoimmune diseases. Therefore, IFN-γ is recognized as one of the important targets for treating autoimmune diseases. Emapalumab, an anti-IFN-γ antibody, was approved by FDA in 2018 for hemophagocytic lymphohistiocytosis, an autoimmune disease that is as refractory as the diseases described above, and is marketed under the trade name Gamifant.

However, as described above, anti-IFN-γ antibodies have problems such as biological contamination and other risks due to being biologics, antigenicity in long-term administration, and conditions for storage and transportation due to being protein preparations. Therefore, the creation of a therapeutic agent that can solve these problems and be effective is desired.

The present inventors attempted to develop an IFN-γ inhibitor using a DNA aptamer as a means to solve the problems described above. The DNA aptamer is a ligand molecule which forms a secondary structure or a tertiary structure of a single-stranded DNA oligonucleotide by forming a complementary strand between complementary sequences in the DNA oligonucleotide molecule, and binds specifically and strongly to a target molecule by its steric structure. The binding of the DNA aptamer can inhibit, suppress or enhance activity of the target molecule. Although DNA aptamers are smaller than antibodies, with a molecular weight of about one-tenth or less than that of antibodies, they have high compatibility and high target selectivity comparable to those of antibodies. Therefore, it is postulated that an IFN-γ inhibitor using a DNA aptamer may be a suitable modality as a means to solve the problem, since the occurrence of adverse effects due to off-target can be minimized and DNA aptamers can be produced through chemical synthesis.

DNA aptamers of the present disclosure have the following advantages and are unexpectedly discovered to be useful: (1) with a relatively small molecular weight, administration by transdermal formulations such as gels is possible; (2) as chemically synthesized products, a risk of biological contamination is low; (3) generally antigenicity is low; (4) due to being DNA, sufficient stability is secured at room temperature under nuclease-free and neutral conditions; and (5) with almost no inhibitory activity to cytochrome P450, a drug-metabolizing enzyme, there is no effect on concomitant agents, and the like. Further, DNA aptamers do not have a problem that an antibody is generated against another antibody, the problem that occurs when long-term treatment with the antibody is necessary, and therefore, long-term administration is possible.

The DNA oligonucleotide of the present embodiment can be used as a DNA aptamer. As a specific means to treat diseases using the DNA aptamer, the means is assumed to treat autoimmune diseases by neutralizing IFN-γ through administration of the aptamer itself or a modified form of the aptamer. In the present embodiment, DNA oligonucleotides listed in Table 1 are used as DNA aptamers.

TABLE 1

| SEQ ID NO. | Sequence (5′ to 3′) |
|---|---|
| 1 | CCCGCCCGGGTCCGCGAAGCGGTAGGTDsTGGGCTA |
| | GGCDsGCTGGCGGG |
| 2 | CCCGCCCGGGTCCGCGAAGCGGTAGGTDsTGGGCTA |
| | GGCDsGCTGGCGGGCGCGAAG |
| 3 | CCCGCCCGGGTCCGCGAAGCGGTAGGTDsTGGGCTA |
| | GGCDsGCTGGCGGGCGCGXAGCG |

The sequence set forth in SEQ ID NO:3 of Table 1 is a sequence in which the 53rd base from the 5′ end of the sequence was replaced with any base X. The X represents any natural base, any non-natural base or a modified base, or a modified base to which a low-molecular-weight compound, a peptide, an oligonucleic acid, an oligosaccharide, a protein or the like, or a high-molecular-weight compound used in vivo (biopolymer) or a biocompatible polymer is bound.

The sequences set forth in SEQ ID NOs: 1-3 of Table 1 include at least one nucleotide having an artificial base Ds. The Ds represents the following chemical strucure:

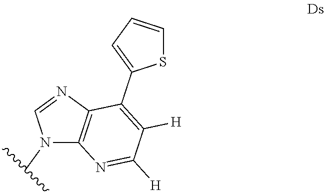 [SMILES: *n1cnc2c(-c3cccs3)ccnc21]

Examples of high-molecular-weight compounds include polyethylene glycols (PEG) with a molecular weight of 20000 or more and any biocompatible large molecules with a molecular weight of 20000 or more. A biocompatible polymer is a chemically synthesized compound that is not normally used in vivo and safe enough to be placed in vivo without causing inflammation or toxic reactions. Examples of medium-molecular-weight compounds include peptides, oligonucleic acids, oligosaccharides, proteins, PEGs, and any biocompatible polymers with a molecular weight of 1000 or more and less than 20000. Examples of low-molecular-weight compounds include antibiotics with a molecular weight of about 200 to 1000.

As functional groups for modification, azido group (—N₃), amino group (—NH₂), carboxyl group (—COOH) or its active ester, alkynyl group (—CC) or a cyclic structure having an alkynyl structure, formyl group (—CHO), hydrazide group (—NH—NH₂), hydroxyl group (—OH), thiol group (—SH), cyano group (—CN), vinyl group (—CHCH₂) and maleimide group can be used.

As used herein, “natural base” refers to either adenine, guanosine, cytosine, or thymine. As used herein, “non-natural base” refers to a base that is artificially synthesized having properties similar to the natural base, and is sometimes referred to as “artificial base” herein. As used herein, “modified base” refers to a base to which a side chain structure with one or more functional groups activated for modification is added, and is a kind of “artificially produced base”. Examples of modifications include methylation, deamination, atomic place exchange, thiolation of oxygen in phosphate site, and introduction of a water-soluble or fat-soluble substituent group into the base portion of a natural base. Specifically, the examples include modified pyrimidines, modified purines, and other heterocyclic bases. Ds in SEQ ID NO: 1 to 3 of Table 1 represents 7-(2-thienyl) imidazo[4,5-b]pyridine, an artificial base. As an artificial base, in addition to Ds itself, a base with a side chain introduced into Ds may be used. Hereafter, in the present embodiment, a DNA aptamer having a sequence set forth in SEQ ID NO:1, 2, or 3 of Table 1 will be referred to as “Aptamer 1”, “Aptamer 2”, or “Aptamer 3”, respectively.

In the present embodiment, it was observed that when a DNA aptamer listed in Table 1 (Aptamer 2) was injected intradermally into the transplanted skin of an immune-tolerance mouse of autoimmune hair loss model which was transplanted with human skin tissue piece, hair loss was suppressed and regeneration of hair once lost was promoted (Example 4). Details will be described later.

Pathological analysis of the mechanism of activity expression of Aptamer 2 or Aptamer 3 in this model revealed that Aptamer 2 or Aptamer 3 almost completely inhibited expression of MHC class I and II. Detailed results will be described later. That is, it is postulated that by inhibiting activity of IFN-γ, Aptamer 2 or Aptamer 3 inhibited production of MHC class I and II, the source of expression of autoimmunity, to improve autoimmunity, thus improving the symptoms of alopecia areata. This indicates that Aptamer 2 or Aptamer 3 is useful as a therapeutic agent not only for alopecia areata but also for other autoimmune diseases (Vasiliki Matzaraki, et. al, Genome Biology, 2017, 18:76; Giulio Cavalli, et. al, Proc. Natl. Acad. Sci. USA, 2016, 113 (5), 1363-1368), and that providing an IFN-γ inhibitor by means of DNA aptamers could be a means to solve the problem.

As a DNA aptamer, DNA oligonucleotides having any of the sequences listed in Table 1 can be used as they are, or it is also possible to use those modified at a site that does not affect activity of the DNA aptamer. Examples of modified forms of DNA aptamers include DNA aptamers bound to medium- or high-molecular-weight compounds such as PEGs, peptides, and oligonucleotides by a chemical method, multimerized DNA aptamers by a chemical method derived from same DNA aptamers, and DNA aptamers in which their sequences are partially converted or modified. When a DNA oligonucleotide of the present embodiment is modified and used as a DNA aptamer, the base portion is preferred as the modification portion. An artificial base or a modified base can be modified using existing methods, and the 3′ and 5′ ends can also be modified.

As drug formulations for systemic administration, formulations can be prepared as an injectable formulation in vials containing lyophilized powders, vials containing aptamer solution, and pre-filled syringes.

The DNA aptamer of the present embodiment can be produced as a preparation for inhalation by placing a nanoparticle adsorbing or containing the DNA aptamer or a solution thereof, or a powder of the DNA aptamer granulated to an appropriate size with a granulating material in an inhalation device.

The DNA aptamer of the present embodiment can be used as an eye drop by dissolving it as it is utilizing its high water-solubility.

As preparations for injection, preparations for inhalation and eye drops, it is possible to use them in a form that the DNA aptamer of the present embodiment is encapsulated or bonded in nanoparticles such as fatty nanoparticles, nanoparticles of biodegradable polymers such as PLGA (Polylactic-co-Glycolic Acid), gold nanoparticles, and then dispersed or dissolved in physiological saline solution, physiological buffer solution, and the like.

In the process of producing a transdermal administration formulation, as an absorption enhancer, lower alcohols such as ethanol, polyhydric alcohols such as ethylene glycol, fatty acids, esters such as ethyl acetate, surfactants, and ionic liquids and the like may be used. For the production of the transdermal administration formulation, production process in which biodegradable polymers such as polylactic acid or liposomes are used for making nanoparticles is applicable, and these processes can be combined as appropriate depending on purposes.

The present disclosure relates to administration of DNA oligonucleotides, such as DNA aptamers, topically into or through the skin, which present unique challenges especially when the underlying diseases or conditions require delivery of therapeutically effective amount of aptamer at various target depths into or through the skin.

### Ionic Liquids

Described herein, in certain embodiments, are compositions comprising an ionic liquid comprising a choline cation and a fatty acid anion. In some embodiments, the composition further comprises a pharmaceutically acceptable solvent. In some embodiments, the fatty acid is myristoleic acid, palmitoleic acid, sapienic acid, oleic acid, elaidic acid, geranic acid, vaccenic acid, linoleic acid, linoelaidic acid, α-linolenic acid, arachidonic acid, eicosapentaenoic acid, erucic acid, docosahexaenoic acid, propionic acid, butyric acid, valeric acid, hexanoic acid, enanthic acid, caprylic acid, pelargonic acid, capric acid, undecylic acid, lauric acid, tridecyclic acid, myristic acid, pentadecylic acid, palmitic acid, margaric acid, stearic acid, nonadecylic acid, arachidic acid, heneicosylic acid, behenic acid, tricosylic acid, lignoceric acid, pentacosylic acid, cerotic acid, heptacosylic acid, montanic acid, nonacosylic acid, melissic acid, henatriacontylic acid, lacceroic acid, psyllic acid, geddic acid, ceroplastic acid, or hexatriacontylic acid. In some embodiments, the fatty acid is geranic acid. In some embodiments, the fatty acid comprises 9 to 14 carbons. In some embodiments, the ionic liquid is liquid at room temperature. In some embodiments, the ionic liquid is liquid below 100° C.

In some embodiments, the ionic liquid is a deep eutectic solvent (DES). In some embodiments, a DES comprises excess carboxylate which precludes 1:1 ion pairing. In some embodiments, a DES further comprises a hydrogen-bond donor. In some embodiments, the hydrogen-bond donor is urea or citric acid. In some embodiments, the solvent properties of a DES are adjusted by changing the hydrogen-bond donor. In some embodiments, the ammonium salt of a DES interacts with a hydrogen-bond donor. In some embodiments, the DES has a melting point lower than either of the individual components (e.g. fatty acid and choline).

In some embodiments, the ionic liquid comprises a molar ratio of a choline cation to a fatty acid anion of 1:0.5 to 1:10. In some embodiments, the molar ratio of the choline cation to the fatty acid anion is about 1:0.5, 1:0.6, 1:0.7, 1:0.8, 1:0.9, 1:1.0; 1:1.1, 1:1.2, 1:1.3, 1:1.4, 1:1.5, 1:1.6, 1:1.7, 1:1.8, 1:1.9, 1:2.0, 1:2.1, 1:2.2, 1:2.3, 1:2.4, 1:2.5, 1:2.6, 1:2.7, 1:2.8, 1:2.9, 1:3.0, 1:3.1, 1:3.2, 1:3.3, 1:3.4, 1:3.5, 1:3.6, 1:3.7, 1:3.8, 1:3.9, 1:4.0, 1:4.1, 1:4.2, 1:4.3, 1:4.4, 1:4.5, 1:4.6, 1:4.7, 1:4.8, 1:4.9. 1:5.0, 1:5.1, 1:5.2, 1:5.3, 1:5.4, 1:5.5, 1:5.6, 1:5.7, 1:5.8, 1:5.9, 1:6.0, 1:6.1, 1:6.2, 1:6.3, 1:6.4, 1:6.5, 1:6.6, 1:6.7, 1:6.8, 1:6.9, 1:7.0, 1:7.1, 1:7.2, 1:7.3, 1:7.4, 1:7.5, 1:7.6, 1:7.7, 1:7.8, 1:7.9, 1:8.0, 1:8.1, 1:8.2, 1:8.3, 1:8.4, 1:8.5, 1:8.6, 1:8.7, 1:8.8, 1:8.9, 1:9.0, 1:9.1, 1:9.2, 1:9.3, 1:9.4, 1:9.5, 1:9.6, 1:9.7, 1:9.8, 1:9.9, or about 1:10. In some embodiments, the molar ratio of the choline cation to the fatty acid anion is about 1:1.1, 1:1.2, 1:1.3, 1:1.4, 1:1.5, 1:1.6, 1:1.7, 1:1.8, 1:1.9, or 1:2.0.

In some embodiments, the choline cation and fatty acid anion are in a molar ratio in the ionic liquid. In some embodiments, the choline cation and fatty acid anion are in a molar ratio of 1:1. In some embodiments, the term Composition B is used herein to refer to a composition or an ionic liquid comprising a 1:1 molar ratio of choline cation to geranic acid anion. In some embodiments, Composition B does not comprise water.

In other embodiments, the choline cation and fatty acid anion are in a molar ratio of 1:2. In some embodiments, the term Composition A is used herein to refer to a composition or an ionic liquid comprising a 1:2 molar ratio of choline cation to geranic acid anion. In some embodiments, Composition A does not comprise water.

In some embodiments, the chemical structure of choline is:

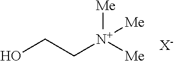

wherein X⁻ is a pharmaceutically acceptable anion.

In some embodiments, term choline refers to the class of quaternary ammonium salts containing the N,N,N-trimethylethanolammonium cation. In some embodiments, the X on the right of the structure of choline denotes a pharmaceutically acceptable anion. In some embodiments the X is bicarbonate, carbonate, acetate, citrate, tartarate, bitartarate, lactate, chloride, bromide, or iodide. In some embodiments, the X″ is bicarbonate. In some embodiments, the choline is an anti-inflammatory agent.

In some embodiments, choline is in the form of a pharmaceutically acceptable salt. The type of pharmaceutical acceptable salts, include, but are not limited to acid addition salts, formed by reacting the free base form of the compound with a pharmaceutically acceptable: inorganic acid such as hydrochloric acid, hydrobromic acid, sulfuric acid, nitric acid, phosphoric acid, metaphosphoric acid, and the like; or with an organic acid such as acetic acid, propionic acid, hexanoic acid, cyclopentanepropionic acid, glycolic acid, pyruvic acid, lactic acid, malonic acid, succinic acid, malic acid, maleic acid, fumaric acid, trifluoroacetic acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, toluenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like.

In some embodiments, the chemical structure of geranic acid, or 3,7-dimethyl-2,6-octadienoic acid, is:

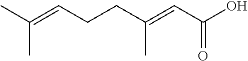

In some embodiments, geranic acid is in the form of a pharmaceutically acceptable salt. The type of pharmaceutical acceptable salts, include, but are not limited to salts formed when an acidic proton present in the parent compound either is replaced by a metal ion, e.g., an alkali metal ion (e.g. lithium, sodium, potassium), an alkaline earth ion (e.g. magnesium, or calcium), or an aluminum ion; or coordinates with an organic base. Examples of acceptable organic bases include, but are not limited to, ethanolamine, diethanolamine, triethanolamine, tromethamine, and N-methylglucamine. Examples of acceptable inorganic bases include, but are not limited to, aluminum hydroxide, calcium hydroxide, potassium hydroxide, sodium carbonate, and sodium hydroxide.

In some embodiments, the choline and the fatty acid are synthesized using any suitable standard synthetic reactions. In some embodiments, the reactions are employed in a linear sequence to provide the compounds or they may be used to synthesize fragments which are subsequently joined by any suitable method. In some embodiments, the starting material used for the synthesis of choline or fatty acid is synthesized or are obtained from commercial sources.

In some embodiments, geranic acid is purified from the commercially available technical grade (Sigma-Aldrich, St. Louis, Mo.) by repeated (5-7×) recrystallization from a solution of 70 wt % geranic acid/30 wt % acetone at −70° C. In some embodiments, purity of the geranic acid is assessed by 1H NMR spectroscopy and conductivity measurements. In some embodiments, the term geranic acid refers to a geranic acid or a salt thereof. In some embodiments, the geranic acid is an anti-microbial agent.

### Topical Gel Composition

In some embodiments, the composition is a topical gel. In some embodiments, the composition is a topical hydrogel.

Gels have been defined in various ways. For example, the United States Pharmacopoeia defines gels as semisolid systems consisting of either suspensions made up of small inorganic particles or large organic molecules interpenetrated by a liquid. Gels include a single-phase or a two-phase system. A single-phase gel consists of organic macromolecules distributed uniformly throughout a liquid in such a manner that no apparent boundaries exist between the dispersed macromolecules and the liquid. Some single-phase gels are prepared from synthetic macromolecules (e.g., carbomer) or from natural gums, (e.g., tragacanth). In some embodiments, single-phase gels are generally aqueous, but will also be made using alcohols and oils. Two-phase gels consist of a network of small discrete particles. The vehicle of an gel is known as the gel base

Gels can also be classified as being hydrophobic or hydrophilic. In certain embodiments, the base of a non-limiting example of a hydrophobic gel includes a liquid paraffin with polyethylene or fatty oils gelled with colloidal silica, or aluminum or zinc soaps. In contrast, the base of a non-limiting example of a hydrophilic gel includes water, glycerol, or propylene glycol gelled with a suitable gelling agent (e.g., tragacanth, starch, cellulose derivatives, carboxyvinylpolymers, and magnesium-aluminum silicates). In certain embodiments, the rheology of the compositions disclosed herein is pseudo plastic, plastic, thixotropic, or dilatant.

In some embodiments, the topical composition is a topical gel, and wherein the topically acceptable carrier comprises water and at least one viscosity-enhancing agent. In some embodiments, the viscosity-enhancing agent is selected from cellulose-based polymers, polyoxyethylene-polyoxypropylene triblock copolymers, dextran-based polymers, polyvinyl alcohol, dextrin, polyvinylpyrrolidone, polyalkylene glycols, chitosan, collagen, gelatin, hyaluronic acid, or combinations thereof.

In some embodiment, the topical gel composition described herein is a semi-solid or id in a gelled state before it is topically administered. For example, suitable viscosity-enhancing agents for such gels include by way of example only, gelling agents and suspending agents. In one embodiment, the enhanced viscosity formulation does not include a buffer. In other embodiments, the enhanced viscosity formulation includes a pharmaceutically acceptable buffer. Sodium chloride or other tonicity agents are optionally used to adjust tonicity, if necessary.

By way of example only, the topically acceptable viscosity agent includes hydroxypropyl methylcellulose, hydroxyethyl cellulose, polyvinylpyrrolidone, carboxymethyl cellulose, polyvinyl alcohol, sodium chondroitin sulfate, sodium hyaluronate. Other viscosity enhancing agents compatible with the targeted ocular site include, but are not limited to, acacia (gum arabic), agar, aluminum magnesium silicate, sodium alginate, sodium stearate, bladderwrack, bentonite, carbomer, carrageenan, Carbopol, xanthan, cellulose, microcrystalline cellulose (MCC), ceratonia, chitin, carboxymethylated chitosan, chondrus, dextrose, furcellaran, gelatin, Ghatti gum, guar gum, hectorite, lactose, sucrose, maltodextrin, mannitol, sorbitol, honey, maize starch, wheat starch, rice starch, potato starch, gelatin, sterculia gum, xanthum gum, gum tragacanth, ethyl cellulose, ethylhydroxyethyl cellulose, ethylmethyl cellulose, methyl cellulose, hydroxyethyl cellulose, hydroxyethylmethyl cellulose, hydroxypropyl cellulose, poly(hydroxyethyl methacrylate), oxypolygelatin, pectin, polygeline, povidone, propylene carbonate, methyl vinyl ether/maleic anhydride copolymer (PVM/MA), poly(methoxyethyl methacrylate), poly(methoxyethoxyethyl methacrylate), hydroxypropyl cellulose, hydroxypropylmethyl-cellulose (HPMC), sodium carboxymethyl-cellulose (CMC), silicon dioxide, polyvinylpyrrolidone (PVP: povidone), Splenda® (dextrose, maltodextrin and sucralose) or combinations thereof. In specific embodiments, the viscosity-enhancing excipient is a combination of MCC and CMC. In another embodiment, the viscosity-enhancing agent is a combination of carboxymethylated chitosan, or chitin, and alginate. The combination of chitin and alginate with the topical agents disclosed herein acts as a controlled release formulation, restricting the diffusion of the topical agents from the formulation. Moreover, the combination of carboxymethylated chitosan and alginate is optionally used to assist in increasing the permeability of the topical agents in the eye.

In one embodiment, the pharmaceutically acceptable enhanced viscosity topically acceptable formulation comprises at least one topical agent and at least one gelling agent. Suitable gelling agents for use in preparation of the gel formulation include, but are not limited to, celluloses, cellulose derivatives, cellulose ethers (e.g., carboxymethylcellulose, ethylcellulose, hydroxyethylcellulose, hydroxymethylcellulose, hydroxypropylmethylcellulose, hydroxypropylcellulose, methylcellulose), guar gum, xanthan gum, locust bean gum, alginates (e.g., alginic acid), silicates, starch, tragacanth, carboxyvinyl polymers, carrageenan, paraffin, petrolatum and any combinations or mixtures thereof. In some other embodiments, hydroxypropylmethylcellulose (Methocel®) is utilized as the gelling agent. In certain embodiments, the viscosity enhancing agents described herein are also utilized as the gelling agent for the gel formulations presented herein.

In some embodiments, the topical gel composition described herein is an in situ gel formulation. In some instances, the in situ gel formation is based on increased pre-corneal residence time of the topical composition which improves ocular bioavailability, corneal mucoadhesion, lysosomal interaction and ionic gelation, improved corneal absorption, thermal gelation, or a combination thereof. In some instances, the in situ gel formulation is activated by pH, temperature, ion, UV, or solvent exchange.

In some instances, the topical gel composition comprises aptamer or pharmaceutically acceptable salt thereof and one or more geling agents. In some instances, the geling agent includes, but is not limited to, poloxamer (e.g. Poloxamer 407), tetronics, ethyl (hydroxyethyl) cellulose, cellulose acetate phthalate (CAP), carbopol (e.g. Carbopol 1342P NF, Carbopol 980 NF), alginates (e.g. low acetyl gellan gum (Gelrite®)), gellan, hyaluronic acid, pluronics (e.g. Pluronic F-127), chitosan, polyvinyl alcohol (PVA), polyvinylpyrrolidone (PVP), dextran, hydroxy propyl methyl cellulose (HPMC), hydroxyethylcellulose (HEC), methylcellulose (MC), thiolated xyloglucan, polymethacrilic acid (PMMA), polyethylene glycol (PEG), pseudolatexes, xyloglucans, or combinations thereof.

In some instances, the in situ gel formation further comprises a permeation enhancer. In some instances, the permeation enhancer includes surfactants (e.g. non-ionic surfactants), benzalkonium chloride, EDTA, surface-active heteroglycosides, calcium chelators, hydroxyl propyl beta cyclodextrin (HP beta CD), bile salts, and the like.

In some embodiments, other gel formulations are useful depending upon the particular topical agent, other pharmaceutical agent or excipients/additives used, and as such are considered to fall within the scope of the present disclosure. For example, other commercially-available glycerin-based gels, glycerin-derived compounds, conjugated, or crosslinked gels, matrices, hydrogels, and polymers, as well as gelatins and their derivatives, alginates, and alginate-based gels, and even various native and synthetic hydrogel and hydrogel-derived compounds are all expected to be useful in the topical agent formulations described herein. In some embodiments, topically acceptable gels include, but are not limited to, alginate hydrogels SAF®-Gel (ConvaTec, Princeton, N.J.), Duoderm® Hydroactive Gel (ConvaTec), Nu-gel® (Johnson & Johnson Medical, Arlington, Tex.); Carrasyn® (V) Acemannan Hydrogel (Carrington Laboratories, Inc., Irving, Tex.); glycerin gels Elta® Hydrogel (Swiss-American Products, Inc., Dallas, Tex.) and K-Y® Sterile (Johnson & Johnson). In further embodiments, biodegradable biocompatible gels also represent compounds present in topically acceptable formulations disclosed and described herein.

In some embodiments, the viscosity-enhancing agent is a cellulose-based polymer selected from cellulose gum, alkylcellulose, hydroxyl-alkyl cellulose, hydroxyl-alkyl alkylcellulose, carboxy-alkyl cellulose, or combinations thereof. In some embodiments, the viscosity-enhancing agent is hydroxyl-alkyl alkylcellulose. In some embodiment, the viscosity-enhancing agent is hydroxypropyl methylcellulose.

### Gel Viscosity

In some embodiments, the composition has a Brookfield RVDV viscosity of from about 10,000 to about 300,000 cps at about 20° C. and sheer rate of 1 s⁻¹. In some embodiments, the composition has a Brookfield RVDV viscosity of from about 15,000 to about 200,000 cps at about 20° C. and sheer rate of 1 s⁻¹. In some embodiments, the composition has a Brookfield RVDV viscosity of from about 50,000 to about 150,000 cps at about 20° C. and sheer rate of 1 s⁻¹. In some embodiments, the composition has a Brookfield RVDV viscosity of from about 70,000 to about 130,000 cps at about 20° C. and sheer rate of 1 s⁻¹. In some embodiments, the composition has a Brookfield RVDV viscosity of from about 90,000 to about 110,000 cps at about 20° C. and sheer rate of 1 s⁻¹.

In some embodiments, the topical gel formulation contains a viscosity enhancing agent sufficient to provide a viscosity of between about 500 and 1,000,000 centipoise, between about 750 and 1,000,000 centipoise; between about 1000 and 1,000,000 centipoise; between about 1000 and 400,000 centipoise; between about 2000 and 100,000 centipoise; between about 3000 and 50,000 centipoise; between about 4000 and 25,000 centipoise; between about 5000 and 20,000 centipoise; or between about 6000 and 15,000 centipoise. In some embodiments, the topical gel formulation contains a viscosity enhancing agent sufficient to provide a viscosity of between about 50,0000 and 1,000,000 centipoise.

In certain embodiments, the viscosity of the gel is in the range from about 1000 to about 2000 cP. In certain embodiments, the viscosity of the gel is in the range from about 1100 to about 1900 cP. In certain embodiments, the viscosity of the gel is in the range from about 1200 to about 1800 cP. In certain embodiments, the viscosity of the gel is in the range from about 1300 to about 1700 cP. In certain embodiments, the viscosity of the gel is in the range from about 1400 to about 1600 cP. In certain embodiments, the viscosity of the gel is about 1500 to about 1600 cP.

In some embodiments, the viscosity of the gel formulations presented herein is measured by any means described. For example, in some embodiments, an LVDV-II+CP Cone Plate Viscometer and a Cone Spindle CPE-40 is used to calculate the viscosity of the gel formulation described herein. In other embodiments, a Brookfield (spindle and cup) viscometer is used to calculate the viscosity of the gel formulation described herein. In some embodiments, the viscosity ranges referred to herein are measured at room temperature. In other embodiments, the viscosity ranges referred to herein are measured at body temperature (e.g., at the average body temperature of a healthy human).

### Gel Dose-To-Dose Uniformity

Typical topical gels are packaged in eye drop bottles and administered as drops. For example, a single administration (i.e. a single dose) of a topical gel may include a single drop, two drops, three drops or more into the eyes of the patient. Furthermore, typical topical gels are packaged in tubes or other squeezable containers with a dispensing nozzle through which strips of the gel are delivered. For example, a single administration (i.e. a single dose) of a topical gel may include a single strip, or multiple strips into the eyes of the patient. In some embodiments, one dose of the topical gel described herein is one drop of the gel composition from the eye drop bottle. In some embodiments, one dose of the topical gel is one strip of the gel composition dispensed through the nozzle of a dispersing tube.

In some cases, described herein include topical gel compositions which provide a dose-to-dose uniform concentrations. In some instances, the dose-to-dose uniform concentration does not present significant viarations of drug content from one dose to another. In some instances, the dose-to-dose uniform concentration does provide consistant drug content from one dose to another.

In some embodiments, the composition has a dose-to-dose topical agent concentration variation of less than 50%. In some embodiments, the composition has a dose-to-dose topical agent concentration variation of less than 40%. In some embodiments, the composition has a dose-to-dose topical agent concentration variation of less than 30%. In some embodiments, the composition has a dose-to-dose topical agent concentration variation of less than 20%. In some embodiments, the composition has a dose-to-dose topical agent concentration variation of less than 10%. In some embodiments, the composition has a dose-to-dose topical agent concentration variation of less than 5%.

In some embodiments, the dose-to-dose topical agent concentration variation is based on 10 consecutive doses. In some embodiments, the dose-to-dose topical agent concentration variation is based on 8 consecutive doses. In some embodiments, the dose-to-dose topical agent concentration variation is based on 5 consecutive doses. In some embodiments, the dose-to-dose topical agent concentration variation is based on 3 consecutive doses. In some embodiments, the dose-to-dose topical agent concentration variation is based on 2 consecutive doses.

A nonsettling formulation should not require shaking to disperse drug uniformly. A “no-shake” formulation is potentially advantageous over formulations that require shaking for the simple reason that patients' shaking behavior is a major source of variability in the amount of drug dosed. It has been reported that patients often times do not or forget to shake their topical compositions that requires shaking before administering a dose, despite the instructions to shake that were clearly marked on the label. On the other hand, even for those patients who do shake the product, it is normally not possible to determine whether the shaking is adequate in intensity and/or duration to render the product uniform. In some embodiments, the topical gel compositions described herein are “no-shake” formulations that maintained the dose-to-dose uniformity described herein.

To evaluate the dose-to-dose uniformity, drop bottles or tubes containing the topical aqueous compositions, the topical gel compositions are stored upright for a minimum of 12 hours prior to the start of the test. To simulate the recommended dosing of these products, predetermined number of drops or strips are dispensed from each commercial bottles or tubes at predetermined time intervals for an extended period of time or until no product was left in the bottle or tube. All drops and strips are dispensed into tared glass vials, capped, and stored at room temperature until analysis. Concentrations of aptamer or pharmaceutically acceptable salt thereof in the expressed drops were determined using a reverse-phase HPLC method.

### Topical Composition Skin Compatibility

In some instances, the composition described herein further comprises a tonicity adjusting agent. Tonicity adjusting agent is an agent introduced into a preparation such as an topical composition to reduce local irritation by preventing osmotic shock at the site of application. In some instances, buffer solution and/or a pH adjusting agent that broadly maintains the topical solution at a particular ion concentration and pH are considered as tonicity adjusting agents. In some cases, tonicity adjusting agents include various salts, such as halide salts of a monovalent cation. In some cases, tonicity adjusting agents include mannitol, sorbitol, dextrose, sucrose, urea, and glycerin. In some instances, suitable tonicity adjustors comprise sodium chloride, sodium nitrate, sodium sulfate, sodium bisulfate, potassium chloride, calcium chloride, magnesium chloride, zinc chloride, potassium acetate, sodium acetate, sodium bicarbonate, sodium carbonate, sodium thiosulfate, magnesium sulfate, disodium hydrogen phosphate, sodium dihydrogen phosphate, potassium dihydrogen phosphate, dextrose, mannitol, sorbitol, dextrose, sucrose, urea, propylene glycol, glycerin, or a combination thereof.

In some cases, the composition described herein further comprises a pH adjusting agent. The pH adjusting agent is used which can be acid or base. The base can be oxides, hydroxides, carbonates, bicarbonates and the likes. The oxides can be metal oxides such as calcium oxide, magnesium oxide and the likes; hydroxides can be of alkali metals and alkaline earth metals such as sodium hydroxide, potassium hydroxide, calcium hydroxide and the likes and carbonates can be sodium carbonate, sodium bicarbonates, potassium bicarbonates and the likes. The acid can be mineral acid and organic acids such as hydrochloric acid, nitric acid, phosphoric acid, acetic acid, citric acid, fumaric acid, malic acid tartaric acid and the likes. In some instances, the pH adjusting agent includes, but is not limited to, acetate, bicarbonate, ammonium chloride, citrate, phosphate, pharmaceutically acceptable salts thereof and combinations or mixtures thereof. In some embodiment, the pH adjusting agent is trolamine.

As described elsewhere herein, the pH of the composition is between about 5.5 and about 8.5. In some instances, the pH of the composition is between about 5.5 and about 8.0. In some instances, the pH of the composition is between about 6.0 and about 8.0. In some instances, the pH of the composition is between about 6.5 and about 8.0. In some instances, the pH of the composition is between about 7.0 and about 8.0. In some instances, the pH of the composition is between about 7.0 and about 7.5. In some instances, the pH of the composition is between about 7.5 and about 8.0.

In some instances, the pH of the composition described herein is about 7.1. In some instances, the pH of the composition described herein is about 7.2. In some instances, the pH of the composition described herein is about 7.3. In some instances, the pH of the composition described herein is about 7.4. In some instances, the pH of the composition described herein is about 7.5. In some instances, the pH of the composition described herein is about 7.6. In some instances, the pH of the composition described herein is about 7.7. In some instances, the pH of the composition described herein is about 7.8. In some instances, the pH of the composition described herein is about 7.9.

In some embodiments, the composition described herein comprises a buffer. In some embodiments, a buffer is selected from borates, borate-polyol complexes, phosphate buffering agents, citrate buffering agents, acetate buffering agents, carbonate buffering agents, organic buffering agents, amino acid buffering agents, or combinations thereof.

In some instances, borates include boric acid, salts of boric acid, other pharmaceutically acceptable borates, and combinations thereof. In some cases, borates include boric acid, sodium borate, potassium borate, calcium borate, magnesium borate, manganese borate, and other such borate salts.

As used herein, the term polyol includes any compound having at least one hydroxyl group on each of two adjacent carbon atoms that are not in trans configuration relative to each other. The polyols can be linear or cyclic, substituted or unsubstituted, or mixtures thereof, so long as the resultant complex is water soluble and pharmaceutically acceptable. In some instances, examples of polyol include: sugars, sugar alcohols, sugar acids and uronic acids. In some cases, polyols include, but are not limited to: mannitol, glycerin, xylitol and sorbitol.

In some embodiments, phosphate buffering agents include phosphoric acid; alkali metal phosphates such as disodium hydrogen phosphate, sodium dihydrogen phosphate, trisodium phosphate, dipotassium hydrogen phosphate, potassium dihydrogen phosphate, and tripotassium phosphate; alkaline earth metal phosphates such as calcium phosphate, calcium hydrogen phosphate, calcium dihydrogen phosphate, monomagnesium phosphate, dimagnesium phosphate (magnesium hydrogen phosphate), and trimagnesium phosphate; ammonium phosphates such as diammonium hydrogen phosphate and ammonium dihydrogen phosphate; or a combination thereof. In some instances, the phosphate buffering agent is an anhydride. In some instances, the phosphate buffering agent is a hydrate.

In some embodiments, borate-polyol complexes include those described in U.S. Pat. No. 6,503,497. In some instances, the borate-polyol complexes comprise borates in an amount of from about 0.01 to about 2.0% w/v, and one or more polyols in an amount of from about 0.01% to about 5.0% w/v.

In some cases, citrate buffering agents include citric acid and sodium citrate.

In some instances, acetate buffering agents include acetic acid, potassium acetate, and sodium acetate.

In some instances, carbonate buffering agents include sodium bicarbonate and sodium carbonate.

In some cases, organic buffering agents include Good's Buffer, such as for example 2-(N-morpholino) ethanesulfonic acid (MES), N-(2-Acetamido)iminodiacetic acid, N-(Carbamoylmethyl) iminodiacetic acid (ADA), piperazine-N,N′-bis(2-ethanesulfonic acid (PIPES), N-(2-acetamido)-2-aminoethanesulfonic acid (ACES), β-Hydroxy-4-morpholinepropanesulfonic acid, 3-Morpholino-2-hydroxypropanesulfonic acid (MOPSO), cholamine chloride, 3-(N-morpholino) propansulfonic acid (MOPS), N,N-bis(2-hydroxyethyl)-2-aminoethanesulfonic acid (BES), 2-[(2-Hydroxy-1,1-bis(hydroxymethyl)ethyl)amino]ethanesulfonic acid (TES), 4-(2-hydroxyethyl)-1-piperazineethanesulfonic acid (HEPES), 3-(N,N-Bis[2-hydroxyethyl]amino)-2-hydroxypropanesulfonic acid (DIPSO), acetamidoglycine, 3-{[1,3-Dihydroxy-2-(hydroxymethyl)-2-propanyl]amino}-2-hydroxy-1-propanesulfonic acid (TAPSO), piperazine-1,4,-bis(2-hydroxypropanesulphonic acid) (POPSO), 4-(2-hydroxyethyl) piperazine-1-(2-hydroxypropanesulfonic acid) hydrate (HEPPSO), 3-[4-(2-hydroxyethyl)-1-piperazinyl]propanesulfonic acid (HEPPS), tricine, glycinamide, bicine or N-tris(hydroxymethyl)methyl-3-aminopropanesulfonic acid sodium (TAPS); glycine; and diethanolamine (DEA).

In some cases, amino acid buffering agents include taurine, aspartic acid and its salts (e.g., potassium salts, etc), E-aminocaproic acid, and the like.

In some instances, the composition described herein further comprises a disinfecting agent. In some cases, disinfecting agents include polymeric biguanides, polymeric quarternary ammonium compounds, chlorites, bisbiguanides, chlorite compounds (e.g. potassium chlorite, sodium chlorite, calcium chlorite, magnesium chlorite, or mixtures thereof), and a combination thereof.

In some instances, the composition described herein further comprises a preservative. In some cases, a perservative is added at a concentration to a composition described herein to prevent the growth of or to destroy a microorganism introduced into the composition. In some instances, microorganisms refer to bacteria (e.g. Proteus mirabilis, Serratia marcesens), virus (e.g. Herpes simplex virus, herpes zoster virus), fungus (e.g. fungi from the genus Fusarium), yeast (e.g. Candida albicans), parasites (e.g. Plasmodium spp., Gnathostoma spp.), protozoan (e.g. Giardia lamblia), nematodes (e.g. Onchocercus volvulus), worm (e.g. Dirofilaria immitis), and/or amoeba (e.g. Acanthameoba).

In some instances, the concentration of the preservative is between about 0.0001% and about 1%, about 0.001% and about 0.8%, about 0.004% and about 0.5%, about 0.008% and about 0.1%, and about 0.01% and about 0.08%. In some cases, the concentration of the preservatives is about 0.001%, 0.002%, 0.003%, 0.004%, 0.005%, 0.006%, 0.008%, 0.009%, 0.009%, 0.01%, 0.015%, 0.02%, 0.025%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9% or 1.0%.

In some embodiments, the preservative is selected from benzalkonium chloride, cetrimonium, sodium perborate, stabilized oxychloro complex, SofZia (Alcon), polyquaternium-1, chlorobutanol, edetate disodium, and polyhexamethylene biguanide.

In some instances, the composition described herein further comprises a stabilizer. Stabilizers that are useful in the topically acceptable formulations disclosed herein include, for example, fatty acids, fatty alcohols, alcohols, long chain fatty acid esters, long chain ethers, hydrophilic derivatives of fatty acids, polyvinyl pyrrolidones, polyvinyl ethers, polyvinyl alcohols, hydrocarbons, hydrophobic polymers, moisture-absorbing polymers, and combinations thereof. In some embodiments, amide analogues of stabilizers are also used. In further embodiments, the chosen stabilizer changes the hydrophobicity of the formulation, improves the mixing of various components in the formulation, controls the moisture level in the formula, or controls the mobility of the phase.

In other embodiments, stabilizers are present in sufficient amounts to inhibit the degradation of the topical agent. Examples of such stabilizing agents, include, but are not limited to: glycerol, methionine, monothioglycerol, EDTA, ascorbic acid, polysorbate 80, polysorbate 20, arginine, heparin, dextran sulfate, cyclodextrins, pentosan polysulfate and other heparinoids, divalent cations such as magnesium and zinc, or combinations thereof.

Additional useful stabilization agents for topically acceptable formulations include one or more anti-aggregation additives to enhance stability of topical formulations by reducing the rate of protein aggregation. The anti-aggregation additive selected depends upon the nature of the conditions to which the topical agents, for example aptamer or pharmaceutically acceptable salt thereof, are exposed. For example, certain formulations undergoing agitation and thermal stress require a different anti-aggregation additive than a formulation undergoing lyophilization and reconstitution. Useful anti-aggregation additives include, by way of example only, urea, guanidinium chloride, simple amino acids such as glycine or arginine, sugars, polyalcohols, polysorbates, polymers such as polyethylene glycol and dextrans, alkyl saccharides, such as alkyl glycoside, and surfactants.

Other useful formulations optionally include one or more topically acceptable antioxidants to enhance chemical stability where required. Suitable antioxidants include, by way of example only, ascorbic acid, methionine, sodium thiosulfate and sodium metabisulfite. In one embodiment, antioxidants are selected from metal chelating agents, thiol containing compounds and other general stabilizing agents.

Still other useful compositions include one or more topically acceptable surfactants to enhance physical stability or for other purposes. Suitable nonionic surfactants include, but are not limited to, polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40.

In other embodiments, an additional surfactant (co-surfactant) and/or buffering agent is combined with one or more of the pharmaceutically acceptable vehicles previously described herein so that the surfactant and/or buffering agent maintains the product at an optimal pH for stability. Suitable co-surfactants include, but are not limited to: a) natural and synthetic lipophilic agents, e.g., phospholipids, cholesterol, and cholesterol fatty acid esters and derivatives thereof; b) nonionic surfactants, which include for example, polyoxyethylene fatty alcohol esters, sorbitan fatty acid esters (Spans), polyoxyethylene sorbitan fatty acid esters (e.g., polyoxyethylene (20) sorbitan monooleate (Tween 80), polyoxyethylene (20) sorbitan monostearate (Tween 60), polyoxyethylene (20) sorbitan monolaurate (Tween 20) and other Tweens, sorbitan esters, glycerol esters, e.g., Myrj and glycerol triacetate (triacetin), polyethylene glycols, cetyl alcohol, cetostearyl alcohol, stearyl alcohol, polysorbate 80, poloxamers, poloxamines, polyoxyethylene castor oil derivatives (e.g., Cremophor® RH40, Cremphor A25, Cremphor A20, Cremophor® EL) and other Cremophors, sulfosuccinates, alkyl sulphates (SLS); PEG glyceryl fatty acid esters such as PEG-8 glyceryl caprylate/caprate (Labrasol), PEG-4 glyceryl caprylate/caprate (Labrafac Hydro WL 1219), PEG-32 glyceryl laurate (Gelucire 444/14), PEG-6 glyceryl mono oleate (Labrafil M 1944 CS), PEG-6 glyceryl linoleate (Labrafil M 2125 CS); propylene glycol mono- and di-fatty acid esters, such as propylene glycol laurate, propylene glycol caprylate/caprate; Brij® 700, ascorbyl-6-palmitate, stearylamine, sodium lauryl sulfate, polyoxethyleneglycerol triiricinoleate, and any combinations or mixtures thereof; c) anionic surfactants include, but are not limited to, calcium carboxymethylcellulose, sodium carboxymethylcellulose, sodium sulfosuccinate, dioctyl, sodium alginate, alkyl polyoxyethylene sulfates, sodium lauryl sulfate, triethanolamine stearate, potassium laurate, bile salts, and any combinations or mixtures thereof; and d) cationic surfactants such as cetyltrimethylammonium bromide, and lauryldimethylbenzyl-ammonium chloride.

In a further embodiment, when one or more co-surfactants are utilized in the topically acceptable formulations of the present disclosure, they are combined, e.g., with a pharmaceutically acceptable vehicle and is present in the final formulation, e.g., in an amount ranging from about 0.1% to about 20%, from about 0.5% to about 10%.

In one embodiment, the surfactant has an HLB value of 0 to 20. In additional embodiments, the surfactant has an HLB value of 0 to 3, of 4 to 6, of 7 to 9, of 8 to 18, of 13 to 15, of 10 to 18.

### Aptamer Topical Composition

In some embodiments, each component in a composition, such as the aptamer, ionic liquid, the pharmaceutically acceptable carrier, and optionally other components, is described a percent (%) of the composition. In some embodiments, the % of the composition is a percent concentration volume/volume (v/v) or a percent concentration weight/volume (w/v).

In some embodiments, the fatty acid is selected from the group consisting of myristoleic acid, palmitoleic acid, sapienic acid, oleic acid, elaidic acid, geranic acid, vaccenic acid, linoleic acid, linoelaidic acid, α-linolenic acid, arachidonic acid, eicosapentaenoic acid, erucic acid, docosahexaenoic acid, propionic acid, butyric acid, valeric acid, hexanoic acid, malonic acid, enanthic acid, caprylic acid, pelargonic acid, capric acid, undecylic acid, lauric acid, tridecyclic acid, myristic acid, pentadecylic acid, palmitic acid, margaric acid, stearic acid, nonadecylic acid, arachidic acid, heneicosylic acid, behenic acid, tricosylic acid, lignoceric acid, pentacosylic acid, cerotic acid, heptacosylic acid, montanic acid, nonacosylic acid, melissic acid, henatriacontylic acid, lacceroic acid, psyllic acid, geddic acid, ceroplastic acid, or hexatriacontylic acid.

In some embodiments, the fatty acid is selected from the group consisting oleic acid, geranic acid, hexanoic acid, and malonic acid. In some embodiments, the fatty acid is geranic acid.

In some embodiments, the topical composition allows therapeutically effective amount of aptamer to reach at least an epidermis layer. In some embodiments, the pharmaceutical composition allows therapeutically effective amount of aptamer to reach at least a dermis layer.

In some embodiments, the pharmaceutical composition allows therapeutically effective amount of aptamer to reach at least a subcutaneous tissue layer. In some embodiments, the pharmaceutical composition allows therapeutically effective amount of aptamer to reach at least a muscle tissue.

In some embodiments, the ionic liquid is a deep eutectic solvent (DES). In some embodiments, the ionic liquid comprises the choline cation and fatty acid anion in a molar ratio in a range of 1:1 to 1:4 of choline cation to fatty acid anion (e.g. geranic acid). In some embodiments, the ionic liquid comprises the choline cation and fatty acid anion in a molar ratio of 1:1 of choline cation to fatty acid anion (e.g. geranic acid). In some embodiments, the ionic liquid comprises the choline cation and fatty acid anion in a molar ratio of 1:2 of choline cation to fatty acid anion (e.g. geranic acid). In some embodiments, the ionic liquid comprises the choline cation and fatty acid anion in a molar ratio of 1:3 of choline cation to fatty acid anion (e.g. geranic acid). In some embodiments, the ionic liquid comprises the choline cation and fatty acid anion in a molar ratio of 1:4 of choline cation to fatty acid anion (e.g. geranic acid).

In some embodiments, the topical composition comprises from about 0.01 wt % to about 5 wt % of an aptamer; from about 0.1 wt % to about 20 wt % of an ionic liquid comprising choline cations and geranic acid anions; and a topically acceptable carrier, wherein topical composition allows a therapeutically effective amount of the aptamer to reach a target depth within or beyond the skin.

In some embodiments, the composition comprises from about 0.01 wt % to about 4 wt % of the aptamer. In some embodiments, the composition comprises from about 0.01 wt % to about 3 wt % of the aptamer. In some embodiments, the composition comprises from about 0.01 wt % to about 2 wt % of the aptamer. In some embodiments, the topical composition comprises from about 0.01 wt % to about 1 wt % of the aptamer. In some embodiments, the composition comprises from about 0.05 wt % to about 1 wt % of the aptamer. In some embodiments, the composition comprises from about 0.1 wt % to about 1 wt % of the aptamer. In some embodiments, the composition comprises from about 0.2 wt % to about 0.8 wt % of the aptamer. In some embodiments, the composition comprises from about 0.3 wt % to about 0.5 wt % of the aptamer, optionally about 0.4 wt %.

In some embodiments, the composition comprises from about 0.1 wt % to about 15 wt % of the ionic liquid. In some embodiments, the composition comprises from about 0.1 wt % to about 10 wt % of the ionic liquid. In some embodiments, the composition comprises from about 0.5 wt % to about 9 wt % of the ionic liquid. In some embodiments, the composition comprises from about 0.5 wt % to about 8 wt % of the ionic liquid. In some embodiments, the composition comprises from about 0.5 wt % to about 7 wt % of the ionic liquid. In some embodiments, the composition comprises from about 0.5 wt % to about 6 wt % of the ionic liquid. In some embodiments, the composition comprises from about 0.5 wt % to about 5 wt % of the ionic liquid. In some embodiments, the composition comprises from about 0.5 wt % to about 4 wt % of the ionic liquid. In some embodiments, the composition comprises from about 0.5 wt % to about 3 wt % of the ionic liquid. In some embodiments, the composition comprises from about 1 wt % to about 3 wt % of the ionic liquid. In some embodiments, the composition comprises about 2 wt % of the ionic liquid.

In some embodiments, the topically acceptable carrier comprises at least one organic solvent. In some embodiment, the topically acceptable carrier is essentially free of monoalcohol solvent. In some embodiments, the at least one organic solvent comprises propylene glycol. In some embodiments, the composition comprises from about 1 wt % to about 30 wt % of propylene glycol. In some embodiments, the composition comprises from about 1 wt % to about 25 wt % of propylene glycol. In some embodiments, the composition comprises from about 1 wt % to about 20 wt % of propylene glycol. In some embodiments, the composition comprises from about 5 wt % to about 20 wt % of propylene glycol. In some embodiments, the composition comprises from about 5 wt % to about 15 wt % of propylene glycol. In some embodiments, the composition comprises from about 5 wt % to about 12 wt % of propylene glycol. In some embodiments, the composition comprises from about 8 wt % to about 12 wt % of propylene glycol. In some embodiments, the composition comprises about 10 wt % of propylene glycol.

In some embodiments, the at least one organic solvent comprises diethylene glycol monoethyl ether. In some embodiments, the composition comprises from about 1 wt % to about 30 wt % of diethylene glycol monoethyl ether. In some embodiments, the composition comprises from about 1 wt % to about 25 wt % of diethylene glycol monoethyl ether. In some embodiments, the composition comprises from about 1 wt % to about 20 wt % of diethylene glycol monoethyl ether. In some embodiments, the composition comprises from about 5 wt % to about 20 wt % of diethylene glycol monoethyl ether. In some embodiments, the composition comprises from about 5 wt % to about 15 wt % of diethylene glycol monoethyl ether. In some embodiments, the composition comprises from about 5 wt % to about 12 wt % of diethylene glycol monoethyl ether. In some embodiments, the composition comprises from about 8 wt % to about 12 wt % of diethylene glycol monoethyl ether. In some embodiments, the composition comprises about 10 wt % of diethylene glycol monoethyl ether.

In some embodiments, the composition further comprises a nonionic surfactant. In some embodiments, the nonionic surfactant comprises polysorbate, preferably polysorbate 80. In some embodiments, the composition comprises from about 0.1 wt % to about 10 wt % of the nonionic surfactant. In some embodiments, the composition comprises from about 0.1 wt % to about 8 wt % of the nonionic surfactant. In some embodiments, the composition comprises from about 0.5 wt % to about 8 wt % of the nonionic surfactant. In some embodiments, the composition comprises from about 0.5 wt % to about 5 wt % of the nonionic surfactant. In some embodiments, the composition comprises from about 0.5 wt % to about 3 wt % of the nonionic surfactant. In some embodiments, the composition comprises from about 1 wt % to about 3 wt % of the nonionic surfactant. In some embodiments, the composition comprises about 2 wt % of the nonionic surfactant. In some embodiment, the composition is essentially free of any ionic surfactant.

In some embodiments, the composition further comprises a humectant. In some embodiments, the humectant comprises glycerin. In some embodiments, the composition comprises from about 1 wt % to about 30 wt % of the humectant. In some embodiments, the composition comprises from about 1 wt % to about 25 wt % of the humectant. In some embodiments, the composition comprises from about 1 wt % to about 20 wt % of the humectant. In some embodiments, the composition comprises from about 5 wt % to about 20 wt % of the humectant. In some embodiments, the composition comprises from about 5 wt % to about 15 wt % of the humectant. In some embodiments, the composition comprises from about 5 wt % to about 12 wt % of the humectant. In some embodiments, the composition comprises from about 8 wt % to about 12 wt % of the humectant. In some embodiments, the composition comprises about 10 wt % of the humectant.

In some embodiments, the composition further comprises a gelling agent. In some embodiments, the gelling agent comprises hydroxyethyl cellulose. In some embodiments, the composition comprises from about 0.1 wt % to about 20 wt % of the humectant. In some embodiments, the composition comprises from about 0.1 wt % to about 15 wt % of the humectant. In some embodiments, the composition comprises from about 0.1 wt % to about 10 wt % of the humectant. In some embodiments, the composition comprises from about 0.1 wt % to about 5 wt % of the humectant. In some embodiments, the composition comprises from about 0.1 wt % to about 2 wt % of the humectant. In some embodiments, the composition comprises from about 0.5 wt % to about 2 wt % of the humectant. In some embodiments, the composition comprises from about 0.5 wt % to about 1 wt % of the humectant. In some embodiments, the composition comprises about 0.8 wt % of the humectant.

In some embodiments, the composition further comprises a preservative. In some embodiments, the preservative is selected from methylparaben, propylparaben, or combination thereof. In some embodiments, the composition comprises from about 0.01 wt % to about 10 wt % of the preservative. In some embodiments, the composition comprises from about 0.01 wt % to about 5 wt % of the preservative. In some embodiments, the composition comprises from about 0.05 wt % to about 5 wt % of the preservative. In some embodiments, the composition comprises from about 0.05 wt % to about 2 wt % of the preservative. In some embodiments, the composition comprises from about 0.05 wt % to about 1 wt % of the preservative. In some embodiments, the composition comprises from about 0.1 wt % to about 1 wt % of the preservative. In some embodiments, the composition comprises from about 0.1 wt % to about 0.5 wt % of the preservative. In some embodiments, the composition comprises about 0.2-0.3 wt % of the preservative.

In some embodiments, the composition further comprises a pH adjusting and/or buffering agent. In some embodiments, the buffering agent comprises trolamine, sodium phosphate dibasic, or both. In some embodiments, the composition comprises from about 0.01 wt % to about 10 wt % of the pH adjusting and/or buffering agent. In some embodiments, the composition comprises from about 0.01 wt % to about 5 wt % of the pH adjusting and/or buffering agent. In some embodiments, the composition comprises from about 0.05 wt % to about 5 wt % of the pH adjusting and/or buffering agent. In some embodiments, the composition comprises from about 0.05 wt % to about 2 wt % of the pH adjusting and/or buffering agent. In some embodiments, the composition comprises from about 0.05 wt % to about 1 wt % of the pH adjusting and/or buffering agent. In some embodiments, the composition comprises from about 0.1 wt % to about 1 wt % of the pH adjusting and/or buffering agent. In some embodiments, the composition comprises from about 0.1 wt % to about 0.5 wt % of the pH adjusting and/or buffering agent. In some embodiments, the composition comprises about 0.2-0.3 wt % of the pH adjusting and/or buffering agent.

In some embodiments, the remaining balance of the composition is water. In some embodiments, the composition is essentially free of water. In some embodiments, the composition comprises from about 50 wt % to about 95 wt % of water. In some embodiments, the composition comprises from about 50 wt % to about 90 wt % of water. In some embodiments, the composition comprises from about 55 wt % to about 90 wt % of water. In some embodiments, the composition comprises from about 55 wt % to about 80 wt % of water. In some embodiments, the composition comprises from about 55 wt % to about 75 wt % of water. In some embodiments, the composition comprises from about 55 wt % to about 70 wt % of water. In some embodiments, the composition comprises from about 55 wt % to about 65 wt % of water. In some embodiments, the composition comprises about 60 wt % to about 65 wt % of water.

In some embodiments, the pharmaceutical composition further comprises a penetration enhancer. In some embodiments, the penetration enhancer is 2-(2-ethoxyethoxy) ethanol or oleyl alcohol. In some embodiments, the penetration enhancer is 2-(2-ethoxyethoxy) ethanol. In some embodiments, the pharmaceutical composition comprises from about 1% to about 20% of 2-(2-ethoxyethoxy) ethanol. In some embodiments, the pharmaceutical composition comprises from about 5% to about 15% 2-(2-ethoxyethoxy) ethanol. In some embodiments, the pharmaceutical composition comprises from about 10% 2-(2-ethoxyethoxy) ethanol.

In some embodiments, the pharmaceutical composition is essentially free of 2-(2-ethoxyethoxy) ethanol. In some embodiments, the pharmaceutical composition is essentially free of oleyl alcohol.

In some embodiments, preparing an ionic liquid comprising a choline cation and a fatty acid anion comprises: (a) mixing choline and a fatty acid in a solvent at room temperature in a predetermined ratio; and (b) removing the solvent in vacuo. In some embodiments, the fatty acid is geranic acid. In some embodiments, the solvent is water. In a particular embodiment, the water is deionized water. In some embodiments, removing the solvent comprises rotary evaporation. In some embodiments, removing the solvent comprises heating the ionic liquid, applying a vacuum to the ionic liquid, or a combination thereof. In some embodiments, preparing the ionic liquid further comprises drying the ionic liquid. In some embodiments, heating the ionic liquid comprises heating the ionic liquid to 60° C. In some embodiments, the heating is done for at least 10 minutes, 20 minutes, 30 minutes, 1 hour, 2 hours, 3 hours, 4 hours, 5 hours, 6 hours, 7 hours, 8 hours, 9 hours, 10 hours, 11 hours, 12 hours, 24 hours, 36 hours, 48 hours or 60 hours. In some embodiments, the vacuum is applied at −100 kPa. In some embodiments, the vacuum is applied for at least 10 minutes, 20 minutes, 30 minutes, 1 hours, 2 hours, 3 hours, 4 hours, 5 hours, 6 hours, 7 hours, 8 hours, 9 hours, 10 hours, 11 hours, 12 hours, 24 hours, 36 hours, 48 hours or 60 hours.

In some embodiments, the ionic liquid has had the solvent used in the ionic liquid preparation process removed. In some embodiments, the ionic liquid does not comprise water.

In some embodiments, choline is choline bicarbonate. In some embodiments, the choline is choline in an 80% wt solution of choline bicarbonate. In some embodiment, the predetermined ratio is a ratio of 1:1, 1:2, 1:3, or 1:4 of a choline cation: fatty acid anion. In one embodiment, the ratio is a molar ratio. In another embodiment, the ratio is ratio by weight.

In some embodiments, isolating the composition further comprises purifying the ionic liquid. In some embodiments, purifying the ionic liquid comprises using conventional techniques, including, but not limited to, filtration, distillation, crystallization, and chromatography. In some embodiments, preparing the ionic liquid further comprises isolating the purified ionic liquid

### Aptamer Topical Delivery

Disclosed herein, in certain embodiments, is a topical composition, comprising aptamer or a pharmaceutically acceptable salt thereof, and an ionic liquid in an amount sufficient to allow a therapeutically effective amount of aptamer to reach a target depth within or beyond the skin, wherein the ionic liquid comprises a choline cation and a fatty acid anion.

Disclosed herein, in certain embodiments, is a method of delivering aptamer to or through a skin, the method comprising administering to the skin a pharmaceutical composition comprising aptamer or a pharmaceutically acceptable salt thereof, and an ionic liquid in an amount sufficient to allow a therapeutically effective amount of aptamer to reach a target depth within or beyond the skin, wherein the ionic liquid comprises a choline cation and a fatty acid anion (e.g. geranic acid).

In some embodiments, the target depth is from about 0.05 mm to about 20 mm. In some embodiments, the target depth is from about 0.1 mm to about 15 mm. In some embodiments, the target depth is from about 1 mm to about 10 mm

In some embodiments, the pharmaceutical composition allows therapeutically effective amount of aptamer to reach at least an epidermis layer. In some embodiments, the amount of aptamer at the epidermis layer is sufficient to inhibit activity of IFN-γ and/or expression of at least one of MHC class I and MHC class II within the epidermis layer.

In some embodiments, the pharmaceutical composition allows therapeutically effective amount of aptamer to reach at least a dermis layer. In some embodiments, the amount of aptamer at the dermis layer is sufficient to inhibit activity of IFN-γ and/or expression of at least one of MHC class I and MHC class II within the dermis layer.

In some embodiments, the pharmaceutical composition allows therapeutically effective amount of aptamer to reach at least a subcutaneous tissue layer. In some embodiments, the amount of aptamer at the subcutaneous tissue layer is sufficient to inhibit activity of IFN-γ and/or expression of at least one of MHC class I and MHC class II within the subcutaneous tissue layer.

In some embodiments, the pharmaceutical composition allows therapeutically effective amount of aptamer to reach at least a muscle tissue. In some embodiments, the amount of aptamer at the muscle tissue is sufficient to inhibit activity of IFN-γ and/or expression of at least one of MHC class I and MHC class II within the muscle tissue.

In some embodiments, the total amount of aptamer reaching at least the epidermis layer provided by the pharmaceutical composition is at least 1.5 times the total amount of aptamer reaching at least the epidermis layer provided by a control composition replacing the ionic liquid with same amount of 2-(2-ethoxyethoxy) ethanol. In some embodiments, the total amount of aptamer reaching at least the epidermis layer provided by the pharmaceutical composition is at least 2 times the total amount of aptamer reaching at least the epidermis layer provided by a control composition replacing the ionic liquid with same amount of 2-(2-ethoxyethoxy) ethanol. In some embodiments, the total amount of aptamer reaching at least the epidermis layer provided by the pharmaceutical composition is at least 2.5 times the total amount of aptamer reaching at least the epidermis layer provided by a control composition replacing the ionic liquid with same amount of 2-(2-ethoxyethoxy) ethanol. In some embodiments, the total amount of aptamer reaching at least the epidermis layer provided by the pharmaceutical composition is at least 3 times the total amount of aptamer reaching at least the epidermis layer provided by a control composition replacing the ionic liquid with same amount of 2-(2-ethoxyethoxy) ethanol.

In some embodiments, the total amount of aptamer reaching at least the epidermis layer provided by the pharmaceutical composition is at least 2 times the total amount of aptamer reaching at least the epidermis layer provided by a control composition comprising a hydrogel and 2% of oleyl alcohol. In some embodiments, the total amount of aptamer reaching at least the epidermis layer provided by the pharmaceutical composition is at least 3 times the total amount of aptamer reaching at least the epidermis layer provided by a control composition comprising a hydrogel and 2% of oleyl alcohol. In some embodiments, the total amount of aptamer reaching at least the epidermis layer provided by the pharmaceutical composition is at least 4 times the total amount of aptamer reaching at least the epidermis layer provided by a control composition comprising a hydrogel and 2% of oleyl alcohol. In some embodiments, the total amount of aptamer reaching at least the epidermis layer provided by the pharmaceutical composition is at least 5 times the total amount of aptamer reaching at least the epidermis layer provided by a control composition comprising a hydrogel and 2% of oleyl alcohol.

In some embodiments, the pharmaceutical composition is administered one or more times a day. In some embodiments, the pharmaceutical composition provides reduced systemic exposure to aptamer as compared to therapeutically effective doses of aptamer through other administrative route.

In some embodiments, the pharmaceutical composition consists essentially of aptamer and the ionic liquid. In some embodiments, the pharmaceutical composition further comprises a pharmaceutically acceptable carrier. In some embodiments, the pharmaceutically acceptable carrier is an aqueous carrier. In some embodiments, the pharmaceutically acceptable carrier comprises a gel base.

In some embodiments, the ionic liquid is present in the pharmaceutical composition at a concentration of from about 1% to about 99%. In some embodiments, the ionic liquid is present in the pharmaceutical composition at a concentration of from about 5% to about 90%. In some embodiments, the ionic liquid is present in the pharmaceutical composition at a concentration of from about 10% to about 80%. In some embodiments, the ionic liquid is present in the pharmaceutical composition at a concentration of from about 15% to about 70%. In some embodiments, the ionic liquid is present in the pharmaceutical composition at a concentration of from about 20% to about 60%. In some embodiments, the ionic liquid is present in the pharmaceutical composition at a concentration of from about 30% to about 50%. In some embodiments, the ionic liquid is present in the pharmaceutical composition at a concentration of about 35% to about 45%. In some embodiments, the ionic liquid is present in the pharmaceutical composition at a concentration of about 40%.

### Skin Penetration and Systemic Exposure

Disclosed herein, in certain embodiments, is a method of locally inhibiting activity of IFN-γ and/or expression of at least one of MHC class I and MHC class II within an epidermis layer, a dermis layer, a subcutaneous tissue layer, or a muscle tissue, the method comprising administering to the skin a pharmaceutical composition comprising aptamer or a pharmaceutically acceptable salt thereof, and an ionic liquid in an amount sufficient to allow a therapeutically effective amount of aptamer to reach a target depth within or beyond the skin, wherein the ionic liquid comprises a choline cation and a fatty acid anion.

In some embodiments, the pharmaceutical composition comprises about 0.01% to about 10% of aptamer or a pharmaceutically acceptable salt thereof. In some embodiments, the pharmaceutical composition comprises about 0.1% to about 8% of aptamer or a pharmaceutically acceptable salt thereof. In some embodiments, the topical gel comprises about 0.1% to about 5% of aptamer or a pharmaceutically acceptable salt thereof.

In some embodiments, the target depth is from about 0.05 mm to about 20 mm. In some embodiments, the target depth is from about 0.1 mm to about 15 mm. In some embodiments, the target depth is from about 1 mm to about 10 mm.

In some embodiments, the pharmaceutical composition allows therapeutically effective amount of aptamer to reach at least the epidermis layer. In some embodiments, the amount of aptamer at the epidermis layer is sufficient to inhibit activity of IFN-γ and/or expression of at least one of MHC class I and MHC class II within the epidermis layer.

In some embodiments, the pharmaceutical composition allows therapeutically effective amount of aptamer to reach at least the dermis layer. In some embodiments, the amount of aptamer at the dermis layer is sufficient to inhibit activity of IFN-γ and/or expression of at least one of MHC class I and MHC class II within the dermis layer.

In some embodiments, the pharmaceutical composition allows therapeutically effective amount of aptamer to reach at least the the subcutaneous tissue layer. In some embodiments, the amount of aptamer at the subcutaneous tissue layer is sufficient to inhibit activity of IFN-γ and/or expression of at least one of MHC class I and MHC class II within the subcutaneous tissue layer.

In some embodiments, the pharmaceutical composition allows therapeutically effective amount of aptamer to reach at least the muscle tissue. In some embodiments, the amount of aptamer at the muscle tissue is sufficient to inhibit activity of IFN-γ and/or expression of at least one of MHC class I and MHC class II within the muscle tissue.

In some embodiments, the total amount of aptamer reaching at least the epidermis layer provided by the pharmaceutical composition is at least 1.5 times the total amount of aptamer reaching at least the epidermis layer provided by a control composition replacing the ionic liquid with same amount of 2-(2-ethoxyethoxy) ethanol. In some embodiments, the total amount of aptamer reaching at least the epidermis layer provided by the pharmaceutical composition is at least 2 times the total amount of aptamer reaching at least the epidermis layer provided by a control composition replacing the ionic liquid with same amount of 2-(2-ethoxyethoxy) ethanol. In some embodiments, the total amount of aptamer reaching at least the epidermis layer provided by the pharmaceutical composition is at least 2.5 times the total amount of aptamer reaching at least the epidermis layer provided by a control composition replacing the ionic liquid with same amount of 2-(2-ethoxyethoxy) ethanol. In some embodiments, the total amount of aptamer reaching at least the epidermis layer provided by the pharmaceutical composition is at least 3 times the total amount of aptamer reaching at least the epidermis layer provided by a control composition replacing the ionic liquid with same amount of 2-(2-ethoxyethoxy) ethanol.

In some embodiments, the total amount of aptamer reaching at least the epidermis layer provided by the pharmaceutical composition is at least 2 times the total amount of aptamer reaching at least the epidermis layer provided by a control composition comprising a Hydrogel and 2% of oleyl alcohol. In some embodiments, the total amount of aptamer reaching at least the epidermis layer provided by the pharmaceutical composition is at least 3 times the total amount of aptamer reaching at least the epidermis layer provided by a control composition comprising a Hydrogel and 2% of oleyl alcohol. In some embodiments, the total amount of aptamer reaching at least the epidermis layer provided by the pharmaceutical composition is at least 4 times the total amount of aptamer reaching at least the epidermis layer provided by a control composition comprising a Hydrogel and 2% of oleyl alcohol. In some embodiments, the total amount of aptamer reaching at least the epidermis layer provided by the pharmaceutical composition is at least 5 times the total amount of aptamer reaching at least the epidermis layer provided by a control composition comprising a Hydrogel and 2% of oleyl alcohol.

In some embodiments, the pharmaceutical composition is administered one or more times a day. In some embodiments, the pharmaceutical composition provides reduced systemic exposure to aptamer as compared to therapeutically effective doses of oral aptamer.

In some embodiments, the fatty acid is selected from the group consisting of myristoleic acid, palmitoleic acid, sapienic acid, oleic acid, elaidic acid, geranic acid, vaccenic acid, linoleic acid, linoelaidic acid, α-linolenic acid, arachidonic acid, eicosapentaenoic acid, erucic acid, docosahexaenoic acid, propionic acid, butyric acid, valeric acid, hexanoic acid, malonic acid, enanthic acid, caprylic acid, pelargonic acid, capric acid, undecylic acid, lauric acid, tridecyclic acid, myristic acid, pentadecylic acid, palmitic acid, margaric acid, stearic acid, nonadecylic acid, arachidic acid, heneicosylic acid, behenic acid, tricosylic acid, lignoceric acid, pentacosylic acid, cerotic acid, heptacosylic acid, montanic acid, nonacosylic acid, melissic acid, henatriacontylic acid, lacceroic acid, psyllic acid, geddic acid, ceroplastic acid, or hexatriacontylic acid. In some embodiments, the fatty acid is selected from the group consisting oleic acid, geranic acid, hexanoic acid, and malonic acid. In some embodiments, the fatty acid is geranic acid.

In some embodiments, the ionic liquid is a deep eutectic solvent (DES). In some embodiments, the ionic liquid comprises the choline cation and fatty acid anion in a molar ratio in a range of 1:1 to 1:4 of choline cation to fatty acid anion. In some embodiments, the ionic liquid comprises the choline cation and fatty acid anion in a molar ratio of 1:2 of choline cation to fatty acid anion.

In some embodiments, the pharmaceutical composition consists essentially of aptamer and the ionic liquid. In some embodiments, the pharmaceutical composition further comprises a pharmaceutically acceptable carrier. In some embodiments, the pharmaceutically acceptable carrier is an aqueous carrier. In some embodiments, the pharmaceutically acceptable carrier comprises an gel base.

In some embodiments, the ionic liquid is present in the pharmaceutical composition at a concentration of from about 1% to about 99%. In some embodiments, the ionic liquid is present in the pharmaceutical composition at a concentration of from about 5% to about 90%. In some embodiments, the ionic liquid is present in the pharmaceutical composition at a concentration of from about 10% to about 80%. In some embodiments, the ionic liquid is present in the pharmaceutical composition at a concentration of from about 15% to about 70%. In some embodiments, the ionic liquid is present in the pharmaceutical composition at a concentration of from about 20% to about 60%. In some embodiments, the ionic liquid is present in the pharmaceutical composition at a concentration of from about 30% to about 50%. In some embodiments, the ionic liquid is present in the pharmaceutical composition at a concentration of about 35% to about 45. In some embodiments, the ionic liquid is present in the pharmaceutical composition at a concentration of about 40%.

In some embodiments, the pharmaceutical composition further comprises a penetration enhancer. In some embodiments, the penetration enhancer is 2-(2-ethoxyethoxy) ethanol or oleyl alcohol. In some embodiments, the penetration enhancer is 2-(2-ethoxyethoxy) ethanol. In some embodiments, the pharmaceutical composition comprises from about 1% to about 20% of 2-(2-ethoxyethoxy) ethanol. In some embodiments, the pharmaceutical composition comprises from about 5% to about 15% 2-(2-ethoxyethoxy) ethanol. In some embodiments, the pharmaceutical composition comprises from about 10% 2-(2-ethoxyethoxy) ethanol.

In some embodiments, the pharmaceutical composition is essentially free of 2-(2-ethoxyethoxy) ethanol. In some embodiments, the pharmaceutical composition is essentially free of oleyl alcohol.

In some embodiments, the amount of the composition administered to the individual and the length of treatment depends on the attributes of the individual including, but not limited to, state of health, weight, severity of the condition, previous therapy, and judgement of the treating physician. In some embodiments, the amount of the composition administered to the individual is determined by routine experimentation (e.g., a dose escalation clinical trial).

In some embodiments, topical compositions as described herein inhibits activity of IFN-γ and/or expression of at least one of MHC class I and MHC class II within the epidermis layer by 10%, 15%, 20%, 25%, 30%, 35%, 40%, 45%, 50%, 55%, 60%, 65%, 70%, 75%, 80%, 85%, 90%, 95%, or more than 95%.

In some embodiments, topical compositions as described herein inhibits activity of IFN-γ and/or expression of at least one of MHC class I and MHC class II within the dermis layer by 10%, 15%, 20%, 25%, 30%, 35%, 40%, 45%, 50%, 55%, 60%, 65%, 70%, 75%, 80%, 85%, 90%, 95%, or more than 95%.

In some embodiments, topical compositions as described herein inhibits activity of IFN-γ and/or expression of at least one of MHC class I and MHC class II within the subcutaneous tissue layer by 10%, 15%, 20%, 25%, 30%, 35%, 40%, 45%, 50%, 55%, 60%, 65%, 70%, 75%, 80%, 85%, 90%, 95%, or more than 95%.

In some embodiments, topical compositions as described herein inhibits activity of IFN-γ and/or expression of at least one of MHC class I and MHC class II within the muscle by 10%, 15%, 20%, 25%, 30%, 35%, 40%, 45%, 50%, 55%, 60%, 65%, 70%, 75%, 80%, 85%, 90%, 95%, or more than 95%.

In some embodiments, the composition is applied to the skin of the individual once a day. In some embodiments, the composition is applied to the skin of the individual 1, 2, 3, 4, or 5 times a day. In some embodiments, the composition is applied to the skin of the individual 2 times a day. In some embodiments, the composition is applied to the skin of the individual 2 times a day, e.g., morning and evening. In some embodiments, the composition is applied to the skin of the individual every day, every other day, every three days, twice a week, once a week, or once a month. In some embodiments, the composition is applied to the skin of the individual once. In some embodiments, the composition is applied to the skin of the individual for a period of time of 1 week, 2 weeks, 3 weeks, 1 month, 2 months, or 3 months or more. In some embodiments, the composition is applied to the skin until the symptoms of the disease or condition associated with activity of IFN-γ and/or expression of at least one of MHC class I and MHC class II, are eliminated. In some embodiments, the composition is applied to the skin until the symptoms of the disease or condition associated with activity of IFN-γ and/or expression of at least one of MHC class I and MHC class II, are reduced.

In some embodiments, the aptamer delivered to the target depth within or beyond the skin remains therapeutically effective for an extended period of time after topical application. In some embodiments, the extended period of time is one month. In some embodiments, the extended period of time is two months. In some embodiments, the extended period of time is three months. In some embodiments, the extended period of time is four months. In some embodiments, the extended period of time is five months. In some embodiments, the extended period of time is six months. In some embodiments, the extended period of time is nine months. In some embodiments, the extended period of time is 1 year. In some embodiments, the extended period of time is 1.5 year. In some embodiments, the extended period of time is 2 years. In some embodiments, the extended period of time is 3 years.

In some embodiments, the topical aptamer composition described herein provides improved stability or less degradation of the aptamer therein. In some embodiments, the topical aptamer composition described herein provides improved stability or less degradation of the aptamer therein. In some embodiments, the topical aptamer composition described herein are stable with respect to compound degradation (e.g. less than 30% degradation, less than 25% degradation, less than 20% degradation, less than 15% degradation, less than 10% degradation, less than 8% degradation, less than 5% degradation, less than 3% degradation, less than 2% degradation, or less than 1% degradation) over a period of any of at least about 1 day, at least about 2 days, at least about 3 days, at least about 4 days, at least about 5 days, at least about 6 days, at least about 1 week, at least about 2 weeks, at least about 3 weeks, at least about 4 weeks, at least about 5 weeks, at least about 6 weeks, at least about 7 weeks, at least about 8 weeks, at least about 3 months, at least about 4 months, at least about 5 months, or at least about 6 months under storage conditions (e.g. room temperature). In other embodiments, the formulations described herein are stable with respect to aptamer degradation over a period of at least about 1 week. Also described herein are formulations that are stable with respect to aptamer degradation over a period of at least about 1 month.

### Dermal Pharmacokinetic/Bioavailability Evaluation of Topical Formulations

Skin is the outermost largest organ in the body. It protects the body from physical, chemical, and microbial assaults and also prevents water loss from the body. The skin comprises three layers, i.e. epidermis, dermis, and subcutaneous tissue. Epidermis is made up of five layers composed of stratum corneum, stratum lucidum, stratum granulosum, stratum spinosum, and stratum basale. The stratum corneum is the outermost layer and comprise dead keratinocytes, and lamellar granules. The corneocytes and lipids form tight junctions in the stratum corneum like brick and mortar. Stratum corneum acts as a permeation and diffusional barrier for topically applied dosage form for skin disorders and transdermal drug delivery. The topical formulations are considered as a mainstay for the treatment of these skin disorders. Topical drug delivery systems are mostly preferred to deliver the drugs to the target site with minimal systemic adverse effects.

The bioavailability of the topical and transdermal administered drugs is affected by various factors. They include permeation through the skin (rate-limiting membrane stratum corneum), physicochemical properties of the drug (pKa, logP, solubility, molecular weight), excipients used in the formulation, type of formulation, presence of enzymes in skin structures, application site, and type of the skin. After the topical or transdermal application of the formulation onto the skin, the drug must permeate deeper layers by crossing various layers. Various permeability enhancement techniques are employed to increase drug transport into the stratum corneum. Consequently, skin cannot be deemed as a single compartment. The estimation of drug retained in skin layers is critical in topical and transdermal drug delivery. Therefore, there is a need to separate the skin into layers to estimate the drug permeation in different layers. Dermato-pharmacokinetic studies can be very useful for evaluation of topical product's performance and assessment in drug permeation-retention in the skin layers.

Many techniques may be used to assess dermato-pharmacokinetics. Parameters such as Cmax, Tmax, and AUC are extensively assessed in dermato-pharmacokinetic studies. Assessment of dermato-pharmacokinetics of administered topical formulations is desirable in appraising the products' safety and efficacy. Several techniques have been explored for the estimation of the drug in the different skin layers. In diseased skin, the stratum corneum barrier function is impaired, which does not hinder drug permeation. For dermato-pharmacokinetic studies, healthy skin may be more recommended than diseased skin. Using two or three techniques simultaneously provides superior benefits. Dermato-pharmacokinetic evaluation can be utilized for quality control tool for complex formulations and can provide the road map for newer product development.

### Dermal Pharmacokinetic/Bioavailability Evaluation Techniques

The dermato-pharmacokinetic method is employed to estimate drug concentrations in the skin layers. Although the stratum corneum might not be the site of action, it is the rate-limiting membrane for drug permeation. The dermato-pharmacokinetics is akin to blood and urine pharmacokinetic studies. The skin kinetics depends on the drug absorption and the termination of administration. Dermato-pharmacokinetics methods assume three mechanisms: i) stratum corneum behavior as the rate-limiting membrane in drug absorption; ii) diffusion of the drug into the dermis is directly proportional to the concentration of drug at the stratum corneum; iii) The drug concentrations in the skin layers (stratum corneum, epidermis, dermis) depict the dermatological efficacy of the drug [Citation2]. The parameters in dermato-pharmacokinetics include Cmax (maximum drug concentration in the skin layers), Tmax (time required to reach Cmax), and Area under the curve (AUC) in stratum corneum, epidermis, dermis. The Cmax, Tmax and AUC parameters are used to evaluate drug molecules' pharmacokinetics in the skin layers [Citation1]. The dermato-pharmacokinetic profile of the topically applied drugs is estimated using in-vitro and in-vivo techniques as discussed in the following section.

### Tape Stripping

Tape stripping is the most widely employed technique for the estimation of the drug in the stratum corneum. The utilization of tape stripping for quantification of drugs in the stratum corneum was proposed in the draft guidance by the United States Department of Health and Human Services in 1998 to assess the bioequivalence of the topical products. This method can be utilized in both in-vitro and in-vivo drug permeation studies for topical delivery systems applied to the skin. It is uncomplicated, easily implemented for quantification of drug retained in the stratum corneum. This technique is minimally invasive in the in-vivo conditions (Preclinical and clinical studies). It involves removing the stratum corneum layer one after another using adhesive tape strips. The tape strip is applied with the pressure using a roller to ensure uniform and proper adhesion onto the skin surface. The tape strip's application pressure should maintain uniform throughout the tape stripping process to get reproducible results. The collected tape strips are added to the solvent of interest in which the drug is highly soluble to extract the drug from the adhesive. The drug extracted is subjected to centrifugation and quantified using a validated analytical technique. The typical limitations with the tape stripping technique is the interference of adhesive, and the number of strips required to remove the stratum corneum is varied from person to person and site of application. There is a requirement of validating the procedure before the utilization of the process in preclinical and clinical studies. The number of stratum corneum layers differs in animals (mice, rat, rabbit, guinea pig, goat, pig) and humans.

The tape stripping technique is validated using the weighing method or the protein estimation method. In weighing method, the tape strip's weight is noted before and after tape stripping, and the protein levels of corneocytes are measured to determine the stratum corneum layers in the skin. Olesen et al. reported that protein removal was decreased with an increase in tape stripping depth. The study results revealed that tape stripping can be used to skin barrier research without discomfort and scars compared to skin biopsies.

### Microdialysis and Open-Flow Microperfusion

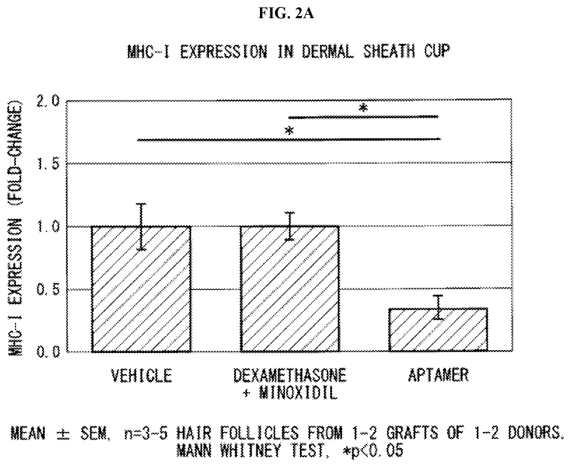

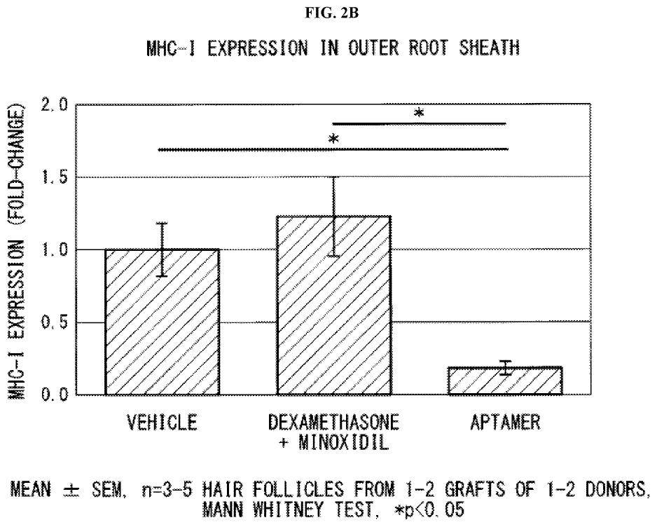

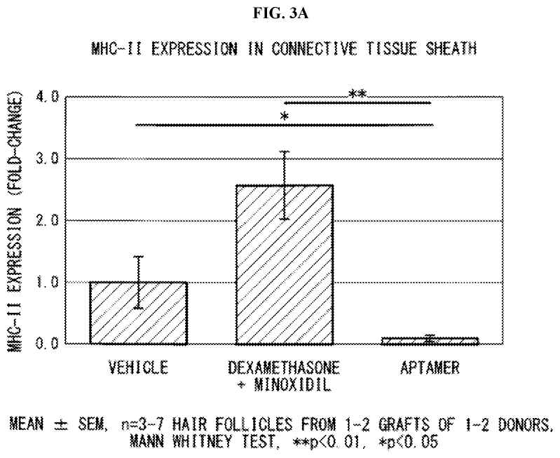

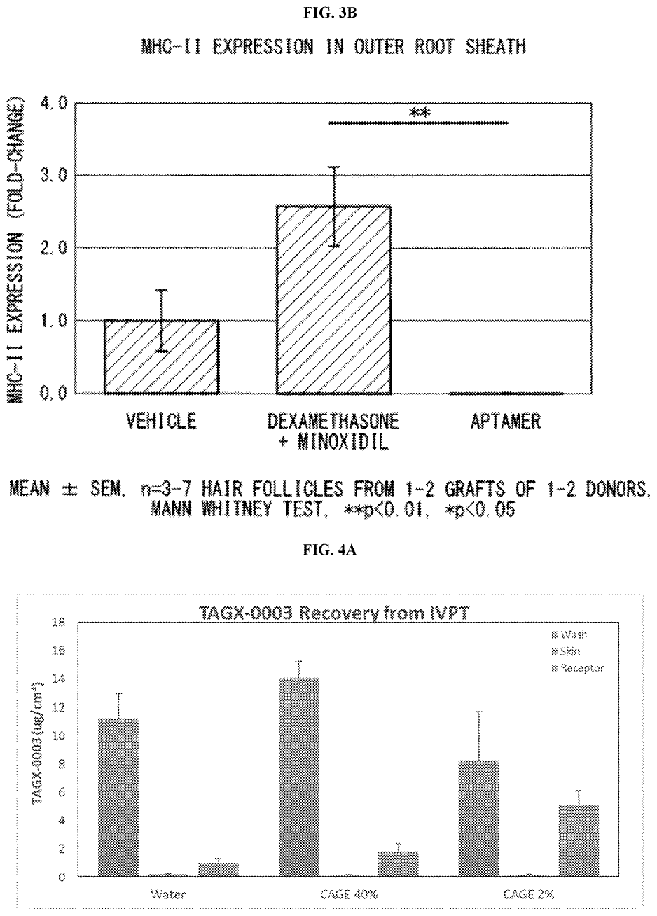

A microdialysis and open-flow microperfusion techniques are employed to assess the bioequivalence of the topical drug product. The drug concentration is determined in the dermis and hypodermis by implanting a semipermeable membrane probe guided by a needle. The probe is perfused with the sterile buffer, which mimics the blood vessels, equilibrates by interstitial fluids, and the drug molecules diffuse from tissues to the buffer. Isotonic saline/Ringer's lactate solution is perfused at the rate of 1-5 μL. FIG. 2 depicts the microdialysis and tape striping technique.

Figure below illustrates microdialysis and tape striping technique: (a) In microdialysis technique, the dialysis tubing is inserted into tissues and physiological solution (saline/ringer solution) is perfused at slow speed. The permeated drug molecules diffuse into the dialysis tube are collected for estimation of drug; (B) in tape stripping technique, the adhesive tape is used to strip the stratum corneum. The adhesive tape strip with stratum corneum is added to the solvent with high drug solubility. The drug is extracted, and the drug is estimated

### Vasoconstrictor Assay

Vasoconstrictor assay is also termed as the skin blanching assay, which is only limited to the corticosteroid formulations' bioavailability assessment. After applying corticosteroids, the local vasoconstriction can be measured by visual examination, chromametry, and analysis using digital imaging. In chromametry, the white light gets reflected into three different constraints red-green, yellow-blue, and light-dark. Using a digital image of 0.5 cm2 with 300 dots per inch is analyzed for the above constraints. Visual inspection is an alternative to chromametry; the visual responses are given in the range of 0-4. Whereas 0 indicates no blanching, and required further no blanching, whereas numbers from 1 to 4 show the increasing array of blanching (absent=0, faint=1, faint-moderate=2, moderate-strong=3, and strong-intense blanching=4) practical application.

### Microscopic and Spectroscopic Methods

Along with the above methods, microscopic and spectroscopic techniques have been widely explored for the assessment of drug distribution. In microscopy analysis techniques, the dye (fluorescein, Nile red, and 5-bromodeoxyuridine, coumarin c6) is utilized for the assessment of the distribution and penetration into deeper layers of the skin. The dye may not exhibit similar behavior as a drug, which is the major limitation of the technique. In spectroscopic techniques, Attenuated Total Reflectance (ATR) or Fourier-Transform Infrared Spectroscopy (FTIR) is employed to estimate the modifications in the stratum corneum modifications. The conformation of proteins in outer skin layers, an assortment of lipids, treatment with chemical enhancers, and employing physical methods may modify permeability. The shift in the infrared bands illustrates the relative alterations between treated and untreated skin. The combination of Raman spectroscopy and confocal microscopy attracted great attention in estimating drug distribution in skin samples. The interference of water in Raman spectroscopy is null; it works on the principle of inelastic scattering of monochromatic light from a laser beam. The chemical structure, electronic configuration, and molecular bonds of detailed compound information are obtained using Raman spectroscopy.

### Dermal Pharmacokinetic/Bioavailability Parameters Estimation

The one compartmental model is employed for estimation of the dermato-pharmacokinetic parameters by the below-mentioned equations:

$${C}_{Skin} = \frac{{K}_{p} \cdot {C}_{max}}{( {K}_{p} - {K}_{e} )} ( {e}^{- Kpt} - {e}^{- Ket} ) \qquad ( 1 )$$

C_{Skin}—Drug concentration in the skin at time t.
   K_{P}—Permeation rate constant (epidermal/dermal).
   Cₘₐₓ—Maximum concentration of drug achieved in the skin layer.
   Kₑ·-Skin elimination rate constant from epidermis/dermis.

$${T}_{max} = \frac{2.303 \times {log}^{Kp / Ke}}{( Kp - Ke )} \qquad ( 2 )$$

Tₘₐₓ—Time required to achieve maximum drug concentration.

$${C}_{max} = \frac{{FX}_{0}}{{V}_{d}} {e}^{- {K}_{e} {T}_{max}} \qquad ( 3 )$$

F—Fraction bioavailable
   Vd—Volume of distribution
   Xo—Initial amount of drug administered

$${AUC}_{( 0 - t )} = {\sum }_{i = 0}^{i = t} \frac{( {C}_{1} + {C}_{2} ) ( {T}_{2} - {T}_{1} )}{2} \qquad ( 4 )$$

AUC—Area under the curve
   T₁ and T₂—Consecutive time points
   C₁ and C₂—Drug concentrations are C₁ and C₂

### NON-LIMITING EMBODIMENTS

The following specific, non-limiting embodiments are to be construed as merely illustrative, and do not limit the present disclosure of the scope of the disclosure. Without further elaboration, it is believed that one skilled in the art can, based on the description herein, utilize the present disclosure to its fullest extent.
1. A topical composition, comprising from about 0.01 wt % to about 5 wt % of an aptamer; from about 0.1 wt % to about 20 wt % of an ionic liquid comprising choline cations and geranic acid anions; and a topically acceptable carrier, wherein topical composition allows a therapeutically effective amount of the aptamer to reach a target depth within or beyond the skin.
2. The topical composition of Embodiment 1, wherein the composition comprises from about 0.01 wt % to about 4 wt % of the aptamer.
3. The topical composition of Embodiment 1, wherein the composition comprises from about 0.01 wt % to about 3 wt % of the aptamer.
4. The topical composition of Embodiment 1, wherein the composition comprises from about 0.01 wt % to about 2 wt % of the aptamer.
5. The topical composition of Embodiment 1, wherein the topical composition comprises from about 0.01 wt % to about 1 wt % of the aptamer.
6. The topical composition of Embodiment 1, wherein the composition comprises from about 0.05 wt % to about 1 wt % of the aptamer.
7. The topical composition of Embodiment 1, wherein the composition comprises from about 0.1 wt % to about 1 wt % of the aptamer.
8. The topical composition of Embodiment 1, wherein the composition comprises from about 0.5 wt % to about 1 wt % of the aptamer, optionally about 0.8 wt %.
9. The topical composition of Embodiment 1, wherein the composition comprises from about 0.3 wt % to about 0.5 wt % of the aptamer, optionally about 0.4 wt %.
10. The topical composition of Embodiment 1-9, wherein the composition comprises from about 0.1 wt % to about 15 wt % of the ionic liquid.
11. The topical composition of Embodiment 1-9, wherein the composition comprises from about 0.1 wt % to about 10 wt % of the ionic liquid.
12. The topical composition of Embodiment 1-9, wherein the composition comprises from about 0.5 wt % to about 9 wt % of the ionic liquid.
13. The topical composition of Embodiment 1-9, wherein the composition comprises from about 0.5 wt % to about 8 wt % of the ionic liquid.
14. The topical composition of Embodiment 1-9, wherein the composition comprises from about 0.5 wt % to about 7 wt % of the ionic liquid.
15. The topical composition of Embodiment 1-9, wherein the composition comprises from about 0.5 wt % to about 6 wt % of the ionic liquid.
16. The topical composition of Embodiment 1-9, wherein the composition comprises from about 0.5 wt % to about 5 wt % of the ionic liquid.
17. The topical composition of Embodiment 1-9, wherein the composition comprises from about 0.5 wt % to about 4 wt % of the ionic liquid.
18. The topical composition of Embodiment 1-9, wherein the composition comprises from about 0.5 wt % to about 3 wt % of the ionic liquid.
19. The topical composition of Embodiment 1-9, wherein the composition comprises from about 1 wt % to about 3 wt % of the ionic liquid.
20. The topical composition of Embodiment 1-9, wherein the composition comprises about 2 wt % of the ionic liquid.
21. The topical composition of Embodiment 1-20, wherein the topically acceptable carrier comprises at least one organic solvent.
22. The topical composition of Embodiment 21, wherein the at least one organic solvent comprises propylene glycol.
23. The topical composition of Embodiment 22, wherein the composition comprises from about 1 wt % to about 30 wt % of propylene glycol.
24. The topical composition of Embodiment 22, wherein the composition comprises from about 1 wt % to about 25 wt % of propylene glycol.
25. The topical composition of Embodiment 22, wherein the composition comprises from about 1 wt % to about 20 wt % of propylene glycol.
26. The topical composition of Embodiment 22, wherein the composition comprises from about 5 wt % to about 20 wt % of propylene glycol.
27. The topical composition of Embodiment 22, wherein the composition comprises from about 5 wt % to about 15 wt % of propylene glycol.
28. The topical composition of Embodiment 22, wherein the composition comprises from about 5 wt % to about 12 wt % of propylene glycol.
29. The topical composition of Embodiment 22, wherein the composition comprises from about 8 wt % to about 12 wt % of propylene glycol.
30. The topical composition of Embodiment 22, wherein the composition comprises about 10 wt % of propylene glycol.
28. The topical composition of Embodiment 21-27, wherein the at least one organic solvent comprises diethylene glycol monoethyl ether.
29. The topical composition of Embodiment 28, wherein the composition comprises from about 1 wt % to about 30 wt % of diethylene glycol monoethyl ether.
30. The topical composition of Embodiment 28, wherein the composition comprises from about 1 wt % to about 25 wt % of diethylene glycol monoethyl ether.
31. The topical composition of Embodiment 28, wherein the composition comprises from about 1 wt % to about 20 wt % of diethylene glycol monoethyl ether.
32. The topical composition of Embodiment 28, wherein the composition comprises from about 5 wt % to about 20 wt % of diethylene glycol monoethyl ether.
33. The topical composition of Embodiment 28, wherein the composition comprises from about 5 wt % to about 15 wt % of diethylene glycol monoethyl ether.
34. The topical composition of Embodiment 28, wherein the composition comprises from about 5 wt % to about 12 wt % of diethylene glycol monoethyl ether.
35. The topical composition of Embodiment 28, wherein the composition comprises from about 8 wt % to about 12 wt % of diethylene glycol monoethyl ether.
36. The topical composition of Embodiment 28, wherein the composition comprises about 10 wt % of diethylene glycol monoethyl ether.
37. The topical composition of Embodiment 1-36, wherein the composition further comprises a nonionic surfactant.
38. The composition of Embodiment 37, wherein the nonionic surfactant comprises polysorbate, preferably polysorbate 80.
39. The topical composition of Embodiment 37-38, wherein the composition comprises from about 0.1 wt % to about 10 wt % of the nonionic surfactant.
40. The topical composition of Embodiment 37-38, wherein the composition comprises from about 0.1 wt % to about 8 wt % of the nonionic surfactant.
41. The topical composition of Embodiment 37-38, wherein the composition comprises from about 0.5 wt % to about 8 wt % of the nonionic surfactant.
42. The topical composition of Embodiment 37-38, wherein the composition comprises from about 0.5 wt % to about 5 wt % of the nonionic surfactant.
43. The topical composition of Embodiment 37-38, wherein the composition comprises from about 0.5 wt % to about 3 wt % of the nonionic surfactant.
44. The topical composition of Embodiment 37-38, wherein the composition comprises from about 1 wt % to about 3 wt % of the nonionic surfactant.
45. The topical composition of Embodiment 37-38, wherein the composition comprises about 2 wt % of the nonionic surfactant.
46. The composition of Embodiment 1-45, wherein the composition further comprises a humectant.
47. The composition of Embodiment 46, wherein the humectant comprises glycerin.
48. The topical composition of Embodiment 46-47, wherein the composition comprises from about 1 wt % to about 30 wt % of the humectant.
49. The topical composition of Embodiment 46-47, wherein the composition comprises from about 1 wt % to about 25 wt % of the humectant.
50. The topical composition of Embodiment 46-47, wherein the composition comprises from about 1 wt % to about 20 wt % of the humectant.
51. The topical composition of Embodiment 46-47, wherein the composition comprises from about 5 wt % to about 20 wt % of the humectant.
52. The topical composition of Embodiment 46-47, wherein the composition comprises from about 5 wt % to about 15 wt % of the humectant.
53. The topical composition of Embodiment 46-47, wherein the composition comprises from about 5 wt % to about 12 wt % of the humectant.
54. The topical composition of Embodiment 46-47, wherein the composition comprises from about 8 wt % to about 12 wt % of the humectant.
55. The topical composition of Embodiment 46-47, wherein the composition comprises about 10 wt % of the humectant.
56. The composition of Embodiment 1-45, wherein the composition further comprises a gelling agent.
57. The composition of Embodiment 46, wherein the gelling agent comprises hydroxyethyl cellulose.
58. The topical composition of Embodiment 56-57, wherein the composition comprises from about 0.1 wt % to about 20 wt % of the gelling agent.
59. The topical composition of Embodiment 56-57, wherein the composition comprises from about 0.1 wt % to about 15 wt % of the gelling agent.
60. The topical composition of Embodiment 56-57, wherein the composition comprises from about 0.1 wt % to about 10 wt % of the gelling agent.
61. The topical composition of Embodiment 56-57, wherein the composition comprises from about 0.1 wt % to about 5 wt % of the gelling agent.
62. The topical composition of Embodiment 56-57, wherein the composition comprises from about 0.1 wt % to about 2 wt % of the gelling agent.
63. The topical composition of Embodiment 56-57, wherein the composition comprises from about 0.5 wt % to about 2 wt % of the gelling agent.
64. The topical composition of Embodiment 56-57, wherein the composition comprises from about 0.5 wt % to about 1 wt % of the gelling agent.
65. The topical composition of Embodiment 56-57, wherein the composition comprises about 0.8 wt % of the gelling agent.
66. The composition of Embodiment 1-65, wherein the composition further comprises a preservative.
67. The composition of Embodiment 66, wherein the preservative is selected from methylparaben, propylparaben, or combination thereof.
68. The topical composition of Embodiment 66-67, wherein the composition comprises from about 0.01 wt % to about 10 wt % of the preservative.
69. The topical composition of Embodiment 66-67, wherein the composition comprises from about 0.01 wt % to about 5 wt % of the preservative.
70. The topical composition of Embodiment 66-67, wherein the composition comprises from about 0.05 wt % to about 5 wt % of the preservative.
71. The topical composition of Embodiment 66-67, wherein the composition comprises from about 0.05 wt % to about 2 wt % of the preservative.
72. The topical composition of Embodiment 66-67, wherein the composition comprises from about 0.05 wt % to about 1 wt % of the preservative.
73. The topical composition of Embodiment 66-67, wherein the composition comprises from about 0.1 wt % to about 1 wt % of the preservative.
74. The topical composition of Embodiment 66-67, wherein the composition comprises from about 0.1 wt % to about 0.5 wt % of the preservative.
75. The topical composition of Embodiment 66-67, wherein the composition comprises about 0.2-0.3 wt % of the preservative.
76. The composition of Embodiment 1-75, wherein the composition further comprises a pH adjusting and/or buffering agent.
77. The composition of Embodiment 76, wherein the pH adjusting and/or buffering agent is selected from trolamine, sodium phosphate dibasic, or both.
78. The topical composition of Embodiment 76-77, wherein the composition comprises from about 0.01 wt % to about 10 wt % of the pH adjusting and/or buffering agent.
79. The topical composition of Embodiment 76-77, wherein the composition comprises from about 0.01 wt % to about 5 wt % of the pH adjusting and/or buffering agent.
80. The topical composition of Embodiment 76-77, wherein the composition comprises from about 0.05 wt % to about 5 wt % of the pH adjusting and/or buffering agent.
81. The topical composition of Embodiment 76-77, wherein the composition comprises from about 0.05 wt % to about 2 wt % of the pH adjusting and/or buffering agent.
82. The topical composition of Embodiment 76-77, wherein the composition comprises from about 0.05 wt % to about 1 wt % of the pH adjusting and/or buffering agent.
83. The topical composition of Embodiment 76-77, wherein the composition comprises from about 0.1 wt % to about 1 wt % of the pH adjusting and/or buffering agent.
84. The topical composition of Embodiment 76-77, wherein the composition comprises from about 0.1 wt % to about 0.5 wt % of the pH adjusting and/or buffering agent.
85. The topical composition of Embodiment 76-77, wherein the composition comprises about 0.2-0.3 wt % of the pH adjusting and/or buffering agent.
86. The composition of Embodiment 1-85, wherein the remaining balance of the composition is water.
87. The topical composition of Embodiment 1-86, wherein the composition comprises from about 50 wt % to about 95 wt % of water.
88. The topical composition of Embodiment 1-86, wherein the composition comprises from about 50 wt % to about 90 wt % of water.
89. The topical composition of Embodiment 1-86, wherein the composition comprises from about 55 wt % to about 90 wt % of water.
90. The topical composition of Embodiment 1-86, wherein the composition comprises from about 55 wt % to about 80 wt % of water.
91. The topical composition of Embodiment 1-86, wherein the composition comprises from about 55 wt % to about 75 wt % of water.
92. The topical composition of Embodiment 1-86, wherein the composition comprises from about 55 wt % to about 70 wt % of water.
93. The topical composition of Embodiment 1-86, wherein the composition comprises from about 55 wt % to about 65 wt % of water.
94. The topical composition of Embodiment 1-86, wherein the composition comprises about 60 wt % to about 65 wt % of water.
95. The topical composition of Embodiment 1-94, wherein the composition is a topical gel.
96. The topical composition of Embodiment 1-94, wherein the composition is a topical hydrogel.
97. The topical composition of any one of Embodiment 1-96, wherein the target depth is from about 0.05 mm to about 20 mm.
98. The topical composition of any one of Embodiment 1-96, wherein the target depth is from about 0.1 mm to about 15 mm.
99. The topical composition of any one of Embodiment 1-96, wherein the target depth is from about 1 mm to about 10 mm.
100. The topical composition of any one of Embodiment 1-99, wherein the pharmaceutical composition allows therapeutically effective amount of aptamer to reach at least an epidermis layer.
101. The topical composition of any one of Embodiment 1-100, wherein the pharmaceutical composition allows therapeutically effective amount of aptamer to reach at least a dermis layer.
102. The topical composition of any one of Embodiment 1-101, wherein the pharmaceutical composition allows therapeutically effective amount of aptamer to reach at least a subcutaneous tissue layer.
103. The topical composition of any one of Embodiments 1-102, wherein the pharmaceutical composition allows therapeutically effective amount of aptamer to reach at least a muscle tissue.
104. The topical composition of any one of Embodiments 1-103, wherein the ionic liquid is a deep eutectic solvent (DES).
105. The topical composition of any one of Embodiments 1-104, wherein the ionic liquid comprises the choline cation and fatty acid anion in a molar ratio in a range of 1:1 to 1:4 of choline cation to geranic acid anion.
106. The topical composition of any one of Embodiments 1-104, wherein the ionic liquid comprises the choline cation and fatty acid anion in a molar ratio of 1:1 choline cation to geranic acid anion.
107. The topical composition of any one of Embodiments 1-104, wherein the ionic liquid comprises the choline cation and fatty acid anion in a molar ratio of 1:2 choline cation to geranic acid anion.
108. The topical composition of any one of Embodiments 1-104, wherein the ionic liquid comprises the choline cation and fatty acid anion in a molar ratio of 1:3 choline cation to geranic acid anion.
109. The topical composition of any one of Embodiments 1-104, wherein the ionic liquid comprises the choline cation and fatty acid anion in a molar ratio of 1:4 choline cation to geranic acid anion.
110. The topical composition of Embodiment 1-109, wherein the aptamer has a nucleotide sequence selected from SEQ ID NO 1-3.
111. The topical composition of Embodiment 1-109, wherein the aptamer has a nucleotide sequence of SEQ ID NO 1.
112. The topical composition of Embodiment 1-109, wherein the aptamer has a nucleotide sequence of SEQ ID NO 2.
113. The topical composition of Embodiment 1-109, wherein the aptamer has a nucleotide sequence of SEQ ID NO 3.
114. The topical composition of Embodiment 1-113, wherein the therapeutically effective amount of the aptamer inhibits IFN-γ.
115. The topical composition of Embodiment 1-114, wherein the therapeutically effective amount of the aptamer inhibits expression of MHC class I.
116. The topical composition of Embodiment 1-115, wherein the therapeutically effective amount of the aptamer inhibits expression of MHC class II.
117. The topical composition of Embodiment 1-116, wherein the therapeutically effective amount of the aptamer restores immune tolerance of hair follicles.
118. The topical composition of Embodiment 1-117, wherein the therapeutically effective amount of the aptamer improves immune tolerance of hair follicles.
119. The topical composition of Embodiment 1-118, wherein the therapeutically effective amount of the aptamer slows progression of alopecia areata.
120. The topical composition of Embodiment 1-119, wherein the therapeutically effective amount of the aptamer arrests progression of alopecia areata.
121. The topical composition of Embodiment 1-160, wherein the therapeutically effective amount of the aptamer reverses alopecia areata.
122. The topical composition of Embodiment 114-121, wherein the aptamer delivered to the target depth within or beyond the skin remains therapeutically effective for an extended period of time after topical application.
123. The topical composition of embodiment 122, wherein the extended period of time is one month.
124. The topical composition of embodiment 122, wherein the extended period of time is two months.
125. The topical composition of embodiment 122, wherein the extended period of time is three months.
126. The topical composition of embodiment 122, wherein the extended period of time is four months.
127. The topical composition of embodiment 122, wherein the extended period of time is five months.
128. The topical composition of embodiment 122, wherein the extended period of time is six months.
129. The topical composition of embodiment 122, wherein the extended period of time is nine months.
130. The topical composition of embodiment 122, wherein the extended period of time is 1 year.
131. The topical composition of embodiment 122, wherein the extended period of time is 1.5 year.
132. The topical composition of embodiment 122, wherein the extended period of time is 2 years.
123. The topical composition of embodiment 122, wherein the extended period of time is 3 years.

### NON-LIMITING EXAMPLES

The following specific, non-limiting examples are to be construed as merely illustrative, and do not limit the present disclosure of the scope of the disclosure. Without further elaboration, it is believed that one skilled in the art can, based on the description herein, utilize the present disclosure to its fullest extent.

### Example 1: Preparation of an Ionic Liquid Containing Choline Cation and Geranic Acid Anion in a 1:1 Molar Ratio (Ionic Liquid B)

The purified GMP Penta Geranic acid (311.0 g, 1.848 mol) was placed in a 2 L round bottomed flask. The flask was placed in a water bath at 20° C. and stirred. Then choline bicarbonate (381.7 g, 1.848 mol) 80% solution in water (Sigma, C7519, 209 ml) was added slowly (drop-wise) with an addition funnel, total addition time was 120 min. The flask was stirred overnight (12 hrs) to maximize the escape of the resulting CO₂. The flask was placed in the rotavap and the remaining CO₂ was removed at room temperature (20° C.) and a small vacuum (30 mbar). After no more CO₂ evolution was observed in the form of foam, the bath was heated to 60° C. and vacuum increased to −100 kPa to remove the resulting water. After no more water evaporation was observed by condensation on the dry ice trap of the rotavap, the flask was further heated at 60° C. and −100 kPa for 36 additional hrs to dry the final product. 475 g of product (94.7% yield) was obtained. HPLC analysis shows 97.9% purity.

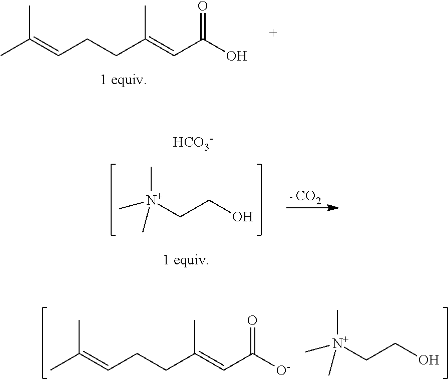

### Example 2: Preparation of an Ionic Liquid Containing Choline Cation and Geranic Acid Anion in a 1:2 Molar Ratio (Ionic Liquid A)

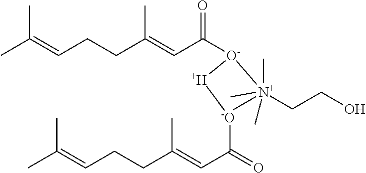

To two equivalents (9.88 g., 0.059 moles) of neat geranic acid, recrystallized 5× at −70° C. from 70% geranic acid/30% acetone, in a 500 mL round bottom flask was added one equivalent of choline bicarbonate (80 wt % solution, 6.06 g, 0.029 mol). The mixture was stirred at room temperature until no more CO₂ evolved. Solvent was removed by rotary evaporation at 60° C. for 20 min, and the product was dried in a vacuum oven for 48 h at 60° C.

Physical characterization at 25° C.: solubility in water=0.5 M; density=0.990 g/mL; conductivity=0.0431 mS/cm; viscosity=1345 cP.

### Example 3: Alternate Preparation of an Ionic Liquid Containing Choline Cation and Geranic Acid Anion in a 1:2 Molar Ratio (Ionic Liquid A)

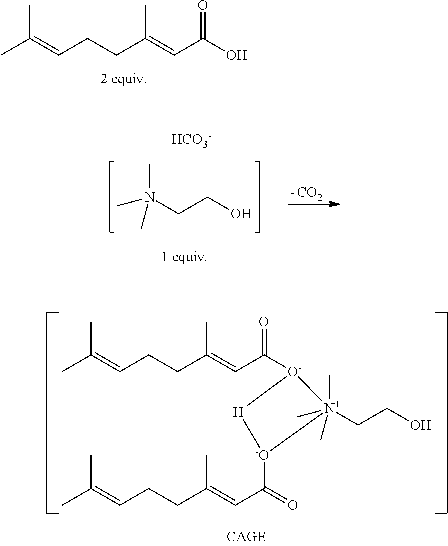

The purified GMP Penta Geranic acid (155 g, 0.921 mol) was placed in a 1 L round bottomed flask. The flask was placed in a water bath at 20° C. and stirred. Then choline bicarbonate (95.1 g, 0.460) 80% solution in water (Sigma, C7519, Lot #: 059K1526V, 209 ml) was added slowly (drop-wise) with an addition funnel, total addition time was 35 min. The flask was stirred overnight (12 hrs) to maximize the escape of the resulting CO₂. The flask was placed in the rotavap and the remaining CO₂ was removed at room temperature (20° C.) and a small vacuum (30 mbar). After no more CO₂ evolution was observed in the form of foam, the bath was heated to 60° C. and vacuum increased to −100 kPa to remove the resulting water. After no more water evaporation was observed by condensation on the dry ice trap of the rotavap, the flask was further heated at 60° C. and −100 kPa for 36 additional hrs to dry the final product. 197 g of Cage (96% yield) was obtained. 1H-NMR spectrum looks similar to the one of CB-0001. HPLC analysis shows 95.1% purity.

### Example 4: Preparation of an Ionic Liquid Containing Choline Cation and Geranic Acid Anion in a 1:3 Molar Ratio

To three equivalents (14.56 g., 0.087 moles) of neat geranic acid, recrystallized 5× at −70° C. from 70% geranic acid/30% acetone, in a 1000 mL round bottom flask is added one equivalent of choline bicarbonate (80 wt % solution, 6.06 g, 0.029 mol). The mixture is stirred at room temperature until no more CO₂ evolved. Solvent is removed by rotary evaporation at 60° C. for 20 min, and the product is dried in a vacuum oven for 48 h at 60° C.

### Example 5: Preparation of an Ionic Liquid Containing Choline and Geranic Acid in a 1:4 Molar Ratio

To four equivalents (19.76 g., 0.118 moles) of neat geranic acid, recrystallized 5× at −70° C. from 70% geranic acid/30% acetone, in a 800 mL round bottom flask is added one equivalent of choline bicarbonate (80 wt % solution, 6.06 g, 0.029 mol). The mixture is stirred at room temperature until no more CO₂ evolved. Solvent is removed by rotary evaporation at 60° C. for 20 min, and the product is dried in a vacuum oven for 48 h at 60° C.

### Example 6: Preparation of Aptamers

The aptamers disclosed herein are chemically synthesized using commercially available solid phase synthesizers at a contract oligonucleotide manufacturing facility in compliance with cGMP regulations.

The process consists of synthesizing the aptamers chemically by solid phase oligonucleotide synthesis using conventional phosphoramidite chemistry and 5′-(4,4′)-dimethoxy-triphenylmethyl (DMTr, trityl) protecting groups. Assembly of an oligonucleotide chain is conducted by the phosphoramidite method on a solid support such as controlled pore glass (CPG) or polystyrene. The assembly is carried out sequentially from the 3′-end towards the 5′-end of the oligonucleotide sequence. Each cycle adds a single nucleoside to the growing oligonucleotide according to the molecular sequence and consists of acidic 5′-deprotection, coupling, oxidation, and capping. Each coupling reaction is carried out by 5-(ethylthio)-1H-tetrazole-activated reaction of the appropriate deoxyribophosphoramidite with the liberated 5′-hydroxyl group of a support-immobilized protected nucleoside or oligonucleotide. After the appropriate number of cycles, the residual 5′-protecting group is removed by acid treatment. The crude aptamers are cleaved from the solid support by treatment with a mixture of ammonia and methylamine, with concomitant removal of the β-cyanoethyl phosphate protecting group and the nucleobase protection groups. This cleavage and deprotection reaction yields the crude oligonucleotide, which is typically purified using preparative strong anion exchange HPLC. The purified aptamers are desalted by ultrafiltration and isolated by freeze drying.

### Example 7 Preparation of a Topical Gel Containing Aptamer and Ionic Liquid (Aptamer+IL Gel)

Aptamer, Ionic Liquid A (according to Example 2), gelling agents, other excipients and water are mixture together until a homogenous composition is achieved. One example of the Aptamer+IL Gel is CGB-600 disclosed herein.

CGB-600 Gel is a viscous transparent colorless to light yellow gel that contains a DNA Aptamer (TAGX-0003) as the active ingredient. The gel formulation was developed to possess desired physical properties, such as appearance, viscosity, and easy spread ability for topical use. The active ingredient in CGB-600 Gel is a DNA Aptamer, TAGX-0003 (i.e. Aptamer 2). It binds to IFNγ receptors to break down the inflammatory pathway that leads to collapse of immune privilege of hair follicles and induces melanogenesis in the epidermis. The aptamer acts extracellularly obviating the need for any transfection agents in the formulation. More importantly, it has a half-life of less than 10 minutes which makes it impractical for a systemic dosage form and an ideal candidate for localized delivery at or near the hair follicle and deep epidermis. TAGX-0003 is generated by the SELEX method against recombinant human IFN-γ and consists of the artificial nucleotide Ds monomer as well as natural bases (A, T, G and C). The Ds monomer is a hydrophobic artificial nucleic base that provides high affinity and specificity against its target. TAGX0003 has a high binding affinity (Kd 33 μM) and specificity to human IFN-γ. The mini-hairpin structure of TAGX-0003 offers increased tolerability to nucleases and serum stability.

### Example 8: Skin Flux Assay of Aptamer Compositions

Aptamer delivery by the following four compositions was measured using a static Franz cell setup (N=3 for each composition).

Study was performed in Franz cells with human cadaver skin clamped over receptor chamber containing saline. The skin sample thickness was between 0.24-0.53 mm. The test composition was applied to surface of skin and occluded. After 24 h at 37° C., the surface of the skin sample was washed with saline and tape stripped to remove compositions remaining on the outer surface of the skin sample. The skin sample was then extracted in methanol: saline, 1:1, v/v. Dermis and epidermis layers were extracted together and not separated. Receptor is the saline underneath the skin that was sampled directly. The concentration of aptamer in these samples was quantified by HPLC.

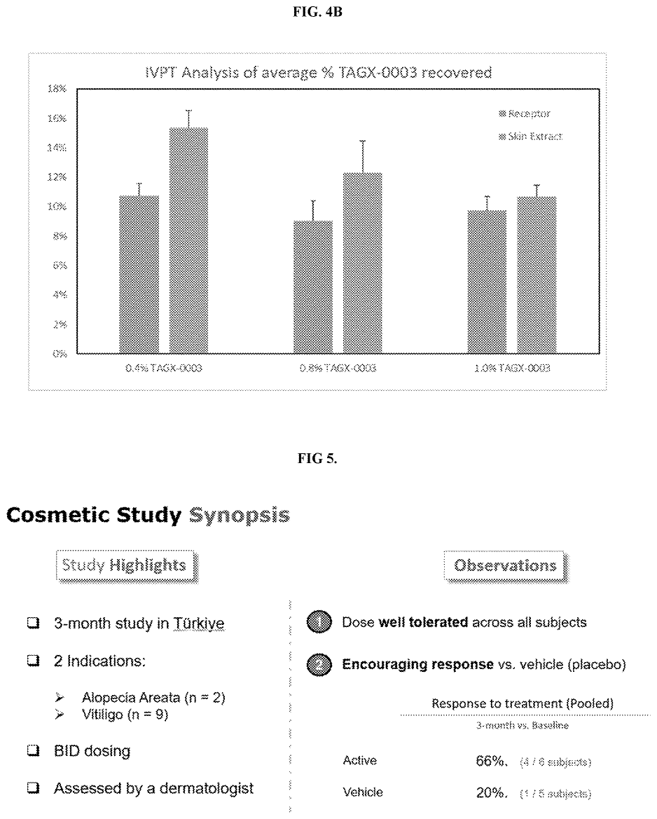

Cumulative skin flux of aptamer was obtained. The data demonstrated that compositions containing ionic liquid according to non-limiting embodiments of the present disclosure resulted in a significantly permeation of aptamer. As shown in FIG. 4A, composition containing 2% of ionic liquid (e.g. choline genranate) provides better dermal bioavailability of the aptamer disclosed herein (e.g. Aptamer 2) than 10% ionic liquid (e.g. choline geranate). As shown in FIG. 4B, composition containing 0.4 wt %, 0.8 wt %, and 1 wt % of the aptamer disclosed herein (e.g. Aptamer 2) provides desirable dermal bioavailability of the aptamer for therapeutic purposes.

### Example 9: Improved Efficacy of Alopecia Areata Treatment by Topical Administration of Aptamer Gel (CGB-600)

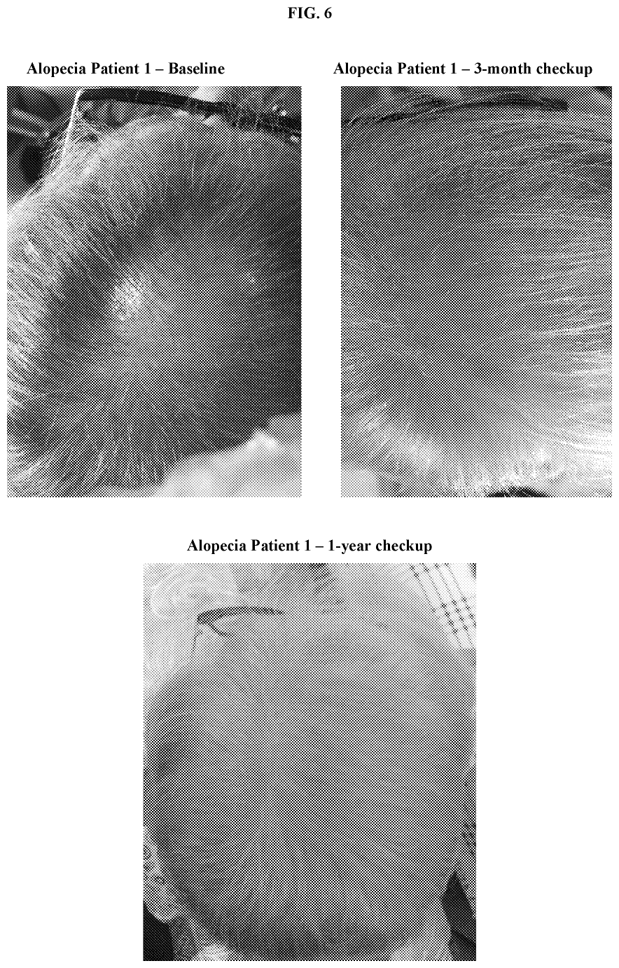

FIG. 5 summarizes the study protocol. FIG. 6 illustrates the result of clinical study on an alopecia areata patient, showing regrowth of hair.

### Example 10: Improved Efficacy of Vitiligo Treatment by Topical Administration of Aptamer Gel (CGB-600)

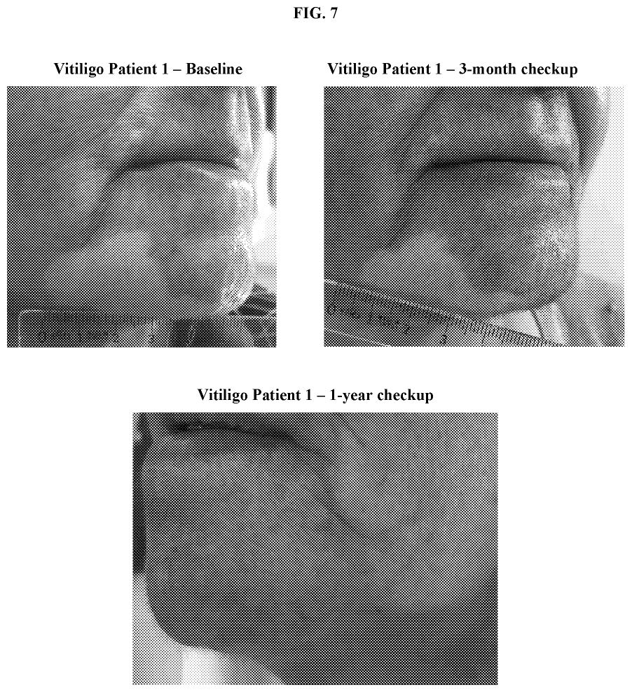

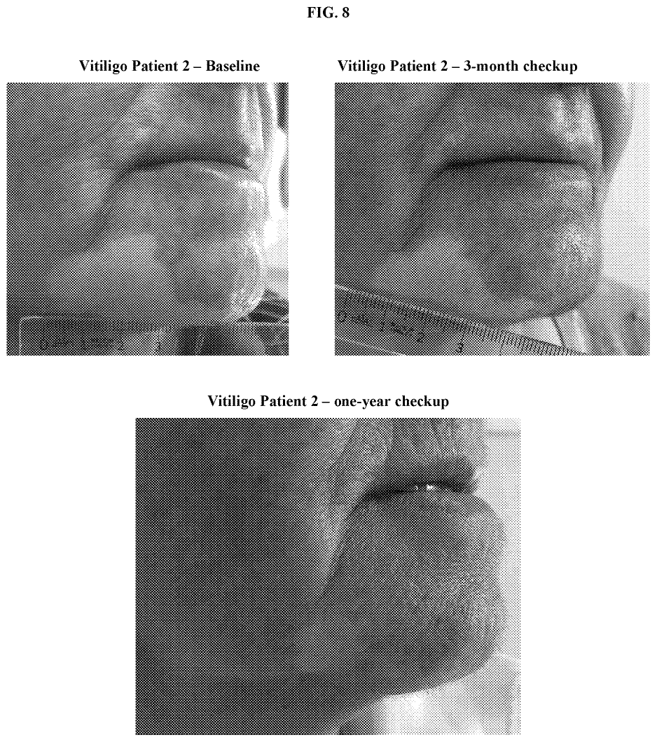

FIG. 5 summarizes the study protocol. FIG. 7 illustrate the result of clinical study on a vitiligo patient, showing re-pigmentation of affect facial areas.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

What is claimed is:

1. A topical composition, comprising:
from about 0.01 wt % to about 5 wt % of an aptamer;
from about 0.1 wt % to about 20 wt % of an ionic liquid comprising choline cations and geranic acid anions;
from about 1 wt % to about 30 wt % of propylene glycol;
from about 1 wt % to about 30 wt % of diethylene glycol monoethyl ether; and
from about 50 wt % to about 95 wt % of water,
wherein topical composition allows a therapeutically effective amount of the aptamer to reach a target depth within or beyond the skin.

2. The topical composition of claim 1, wherein the aptamer comprises TAGX-0003.

3. The topical composition of claim 1, wherein the composition comprises from about 0.1 wt % to about 2 wt % of the aptamer.

4. The topical composition of claim 1, wherein the composition comprises from about 0.5 wt % to about 1 wt % of the aptamer.

5. The topical composition of claim 1, wherein the composition further comprises from about 0.5 wt % to about 5 wt % of a nonionic surfactant.

6. The topical composition of claim 5, wherein the nonionic surfactant comprises polysorbate 80.

7. The topical composition of claim 1, wherein the composition comprises from about 0.5 wt % to about 5 wt % of the ionic liquid.

8. The topical composition of claim 1, wherein the composition comprises from about 0.5 wt % to about 3 wt % of the ionic liquid.

9. The topical composition of claim 1, wherein the composition comprises from about 1 wt % to about 3 wt % of the ionic liquid.

10. The topical composition of claim 1, wherein the composition comprises from about 5 wt % to about 15 wt % of propylene glycol.

11. The topical composition of claim 1, wherein the composition comprises from about 8 wt % to about 12 wt % of propylene glycol.

12. The topical composition of claim 1, wherein the composition comprises from about 5 wt % to about 15 wt % of diethylene glycol monoethyl ether.

13. The topical composition of claim 1, wherein the composition comprises from about 8 wt % to about 12 wt % of diethylene glycol monoethyl ether.

14. The topical composition of claim 1, further comprising from about 1 wt % to about 30 wt % of glycerin.

15. The topical composition of claim 14, wherein the composition comprises from about 5 wt % to about 15 wt % of glycerin.

16. The topical composition of claim 15, wherein the composition comprises from about 8 wt % to about 12 wt % of glycerin.

17. The topical composition of claim 1, further comprising from about 0.1 wt % to about 10 wt % of hydroxyethyl cellulose.

18. The topical composition of claim 17, wherein the composition comprises from about 0.5 wt % to about 2 wt % of hydroxyethyl cellulose.

18. The topical composition of claim 1, wherein the composition further comprises methylparaben and/or propylparaben.

19. The topical composition of claim 1, wherein the composition further comprises trolamine and/or sodium phosphate dibasic.

20. The topical composition of claim 2, wherein the therapeutically effective amount of the aptamer slows progression of, arrests, or reverses, alopecia areata, and wherein optionally the aptamer delivered to the target depth within or beyond the skin remains therapeutically effective for an extended period of time after topical application, wherein the extended period of time is at least one month.

21. The topical composition of claim 2, wherein the therapeutically effective amount of the aptamer slows progression of, arrests, or reverses, vitiligo, and wherein optionally the aptamer delivered to the target depth within or beyond the skin remains therapeutically effective for an extended period of time after topical application, wherein the extended period of time is at least one month.
